(19) United States
(12) Patent Application Publication
(10) Pub. No.: US 2011/0104167 A1
(43) Pub. Date: May 5, 2011

(54) Compositions and Methods for Diagnosing and Treating Insulin Resistance

(75) Inventors: Yehiel Zick, Carmei-Yosef (IL); Yanfang Liu, Lin Fen (CN); Denise Ronen, Yavne (IL)

(73) Assignee: YEDA RESEARCH AND DEVELOPMENT CO. LTD., Rehovot (IL)

(21) Appl. No.: 11/666,946

(22) PCT Filed: Oct. 30, 2005
(86) PCT No.: PCT/IL2005/001131

§ 371 Date: Feb. 9, 2009

Related U.S. Application Data
(60) Provisional application No. 60/622,910, filed on Oct. 29, 2004.

(51) Int. Cl. A61K 39/395; A61K 38/17; A61K 31/7088; A61K 35/12; C07K 14/00; C07H 21/00; A61P 3/10; C12N 5/10; G01N 33/53

(52) U.S. Cl. 424/139.1; 514/6.7; 514/44 R; 424/93.7; 530/300; 536/23.5; 435/361; 436/86; 435/7.1; 436/501

(57) ABSTRACT

A method of treating insulin resistance in a subject is provided. The method comprising administering to the subject a therapeutically effective amount of an agent capable of down-regulating phosphorylation of an IRS protein at at least one serine residue corresponding to amino acid 341, 412 and/or 413 of human IRS-1, thereby treating the insulin resistance in the subject. Also provided are methods and kits for diagnosing insulin resistance.

### FIELD AND BACKGROUND OF THE INVENTION

The present invention relates to compositions, methods and kits for diagnosing and treating insulin resistance, such as that associated with Type II diabetes.

Insulin resistance is a state in which target cells fail to respond to normal levels of circulating insulin. See, e.g. Saltiel et al., Nature 414: 799-806 (2001). The lack of response to insulin, in turn, results in hyperinsulinemia to compensate for the resistance to insulin in the prediabetic state. Subsequently, hyperglycemia develops due to the failure of the pancreatic beta cells to produce and secrete enough insulin to compensate for the imbalance in glucose metabolism. Insulin resistance is commonly associated with prevalent type 2 diabetes. Type 2 diabetes is the most common form of the disease, affecting 16 million people in the United States alone. (Source: American Diabetes Association, www.diabetes.org). Roughly one-third of these people remain undiagnosed.

At the molecular level, insulin resistance may result from mutations or posttranslational modifications of the insulin receptor or any of its downstream targets. The identification of simple molecular explanations for insulin resistance and type 2 diabetes has so far proven difficult. Understanding the basic mechanisms of insulin resistance at the molecular level could have a great impact on finding a cure for a chronic disease like type-2 diabetes and insulin resistance that develops in other conditions such as chronic obesity and acute trauma.

The insulin receptor (IR) mediates insulin action through the phosphorylation of substrate proteins on Tyr residues. IR substrates include the three isoforms of Shc, IRS proteins (IRS-1 to -4), p60dok , Cbl, APS, and Gab-1 {reviewed in (16, 20, 29, 40)}. IRS proteins contain a conserved pleckstrin homology (PH) domain, located at their amino terminus, that serves to anchor the IRS proteins to membrane phosphoinositides in close proximity to the insulin receptor (35). The PH domain of IRS proteins is flanked by a P-Tyr binding (PTB) domain. The PTB domain, present in a number of signaling molecules (24), shares 75% sequence identity between IRS-1 and IRS-2 (31) and functions as a binding site to the NPXY motif at the juxtamembrane (JM) domain of the insulin receptor (7, 36). The C-terminal region of IRS proteins is poorly conserved. It contains multiple Tyr phosphorylation motifs that serve as a signaling scaffold, providing a docking interface for SH2 domain-containing proteins like the p85α regulatory subunit of PI3K, Grb2, Nck, Crk, Fyn and SHP-2, which further propagate the metabolic and growth-promoting effects of insulin (16, 20, 29, 40).

IRS-1 contains over 70 potential Ser/Thr phosphorylation sites with homologies to consensus phosphorylation sites for casein kinase II, PKB, PKC, MAP kinases, CDC2, cAMP- and cGMP-dependent protein kinase (33). Phosphorylation of Ser/Thr residues of IRS proteins has a dual function and serves either for a positive or for a negative modulation of insulin signal transduction. Ser phosphorylation, within the PTB domain of IRS-1, by insulin-stimulated PKB, protects IRS proteins from the rapid action of protein tyrosine phosphatases and enables them to maintain their Tyr-phosphorylated active conformation, implicating PKB as a positive regulator of IRS-1 functions (26). By contrast, Ser/Thr phosphorylation of IRS proteins by other insulin-stimulated Ser/Thr kinases such as PKC (22) serves as a physiological negative feed-back control mechanism, utilized by insulin, to uncouple IR-IRS complexes, inhibit further Tyr phosphorylation of IRS proteins, and terminate insulin signaling. Furthermore, inducers of insulin resistance such as free-fatty acids take advantage of this physiological shut-off mechanism and activate Ser/Thr kinases that phosphorylate the IRS-1 at the same inhibitory sites (reviewed in (40)}.

Ser/Thr phosphorylation can induce the dissociation of IRS proteins from the insulin receptor (IR) (14, 22, 25); hinder Tyr phosphorylation sites (23); release the IRS proteins from intracellular complexes that maintain them in close proximity to the receptor (34); induce IRS proteins degradation (27); or turn IRS proteins into inhibitors of the IRK (15). These multiple effects suggest that the Ser sites subjected to phosphorylation play a key role in regulating IRS-1 function. Several such Ser residues were identified. Ser307, the phosphorylation of which is catalyzed by a number of kinases (1, 10, 39), negatively regulates IRS functions. Because Ser307 is adjacent to the PTB domain of IRS-1, its phosphorylation might disrupt the interaction between the juxtamembrane domain of the IR and the PTB domain IRS-1 and in such a way, inhibit insulin-stimulated Tyr-phosphorylation of IRS-1. Similarly, ‘conventional’ members of the PKC family which are activated by phorbol esters or endothelin-1, stimulate members of the MAPK pathway to phosphorylate IRS-1 at Ser612 and at additional sites in its COOH tail (5). Such phosphorylation inhibits the interactions of IRS-1 both with IR and with downstream effectors of IRS-1, such as PI3K.

Accordingly U.S. Pat. Publ. No. 20040097713 teaches phosphorylation of IRS1/2 at Serine 1101 and 1149 and use of antibodies directed thereagainst for diagnosing insulin resistance.

Still, phosphorylation of IRS-1 at the above sites cannot account for all the effects of Ser kinases on IRS proteins. There is thus a widely recognized need for the identification of novel Ser sites that undergo insulin-dependent phosphorylation by IRS kinases, thereby negatively regulating IRS-1 function.

### SUMMARY OF THE INVENTION

According to one aspect of the present invention there is provided a method of treating insulin resistance in a subject, the method comprising administering to the subject a therapeutically effective amount of an agent capable of down-regulating phosphorylation of an IRS protein at at least one serine residue corresponding to amino acid 341, 412 and/or 413 of human IRS-1, thereby treating the insulin resistance in the subject.

According to further features in preferred embodiments of the invention described below, the insulin resistance is associated with a disease or condition selected from the group consisting of Type II diabetes, obesity, hyperglycemia and hyperlipidemia.

According to still further features in the described preferred embodiments the agent is selected from the group consisting of: (i) a peptide comprising an IRS amino acid sequence not exceeding 50 amino acids in length which comprises the at least one serine residue; (ii) an isolated polynucleotide which comprises a nucleic acid sequence encoding an IRS protein which comprises a mutation in the at least one serine residue; and (iii) cells expressing the isolated polynucleotide of (ii).

According to still further features in the described preferred embodiments the IRS protein is selected from the group consisting of IRS-1, IRS-2 and IRS-4.

According to another aspect of the present invention there is provided a peptide comprising an IRS amino acid sequence not exceeding 50 amino acids in length which comprises at least one serine residue corresponding to amino acid 341, 412 and/or 413 of human IRS-1.

According to yet another aspect of the present invention there is provided an antibody comprising an antigen recognition domain capable of specifically binding an IRS protein phosphorylated on at least one serine residue corresponding to amino acid 341, 412 and/or 413 of human IRS-1 but does not bind the IRS protein when not phosphorylated on this respective position.

According to still another aspect of the present invention there is provided an isolated polynucleotide comprising a nucleic acid sequence encoding an IRS protein which comprises a mutation in at least one serine residue corresponding to amino acid 341, 412 and/or 413 of human IRS-1.

According to an additional aspect of the present invention there is provided a nucleic acid construct comprising the isolated polynucleotide.

According to yet an additional aspect of the present invention there is provided a cell comprising the isolated polynucleotide.

According to still an additional aspect of the present invention there is provided a method of diagnosing insulin resistance in a subject, the method comprising detecting in a biological sample of the subject, presence, absence or level of an IRS protein phosphorylated on at least one serine residue corresponding to amino acid 341, 412 and/or 413 of human IRS-1, wherein the presence or level of the phosphorylated IRS protein is indicative of the insulin resistance in the subject.

According to still further features in the described preferred embodiments the detecting the presence, absence or level of phosphorylated IRS protein is effected via the antibody of claim 6.

According to still further features in the described preferred embodiments the biological sample is a blood sample, a liver sample and/or an adipose tissue derived sample.

According to a further aspect of the present invention there is provided a method of identifying agents suitable for treating insulin resistance, the method comprising: (a) contacting a biological sample comprising an IRS phosphorylated on at least one serine residue corresponding to amino acid 341, 412 and/or 413 of human IRS-1 with a plurality of agents; and (b) identifying at least one agent of the plurality of agents being capable of dephosphorylating the at least one serine residue, thereby identifying agents suitable for treating insulin resistance.

According to yet a further aspect of the present invention there is provided a kit for diagnosing insulin resistance in a subject, the kit comprising a packaging material packaging the antibody.

According to still a further aspect of the present invention there is provided a pharmaceutical composition comprising as an active ingredient the isolated polynucleotide and a pharmaceutically acceptable carrier or diluent.

According to still a further aspect of the present invention there is provided a pharmaceutical composition comprising as an active ingredient the isolated polynucleotide and a pharmaceutically acceptable carrier or diluent.

According to still a further aspect of the present invention there is provided a pharmaceutical composition comprising as an active ingredient the cell and a pharmaceutically acceptable carrier or diluent.

The present invention successfully addresses the shortcomings of the presently known configurations by providing compositions, methods, compositions and kits for diagnosing and treating insulin resistance.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c show Tyrosine (Tyr) phosphorylation of IRS-1^{7A} and its interactions with IR. FIG. 1a—monolayers of CHO-T cells stably overexpressing either WT-IRS-1 (CHO-T^{WT}) or IRS-1^{7A} (CHO-T^{7A}) at 80% confluence (grown in 6 cm plates) were deprived of serum for 16 h prior to the experiment. The cells were then incubated with insulin for the indicated times at 37° C. Cells extracts (100 μg) were resolved on 7.5 SDS-PAGE and immunoblotted with the indicated antibodies. The results are of a representative of six experiments. FIG. 1b—IRS-1 was isolated from CHO-T^{WT} or CHO-T^{7A} cells. In vitro phosphorylation of IRS-1^{WT} and IRS-1^{7A} by Insulin receptor (IR) was effected as described under “Materials and Experimental Procedures” section of the Examples section which follows. The reaction mixture was subjected to immunoprecipitation with anti-IRS-1 antibodies. Immunocomplexes were resolved on 7.5 SDS-PAGE and immunoblotted with anti-phosphotyrosine (pY) or anti IRS-1 antibodies. FIG. 1c—CHO-T^{WT} (expressing wild type IRS-1) and CHO-T^{7A} (expressing the 7A IRS-1 mutant) cells were deprived of serum for 16 h prior to the experiment. The cells were stimulated with 100 nM insulin for the indicated times at 37° C. Cell extracts were prepared and samples (1 mg) were bound to immobilized IR. IRS-1-IR complexes were resolved by means of 7.5% SDS-PAGE and were immunoblotted with the indicated antibodies. In parallel, samples of total cell extracts (100 μg) were resolved on 7.5% SDS-PAGE and immunoblotted with anti-IRS-1 antibodies. The results are of a representative of two experiments performed in duplicates.

FIGS. 2a-b show overexpression of Myc-IRS-1^{WT} and Myc-IRS-1^{7A} in Fao cells. Monolayers of Fao cells at 70-80% confluence (grown in 6 cm plates) were infected with Ad-Myc-IRS-1^{WT} or Ad-Myc-IRS-1^{7A} at the indicated PFUs (FIG. 2a), or with an insert-free adenoviral construct at 7×10⁷ PFU/ml (FIG. 2b). Following 48 hours, cells were remained untreated (FIG. 2a) or were treated with insulin for the indicated times (FIG. 2b). Cell extracts were prepared, samples were resolved on 7.5% SDS-PAGE and were immunoblotted with the indicated antibodies. Results are of a representative of two (FIG. 2b) or three (FIG. 2a) similar experiments.

FIGS. 3a-b show Insulin-induced Tyr phosphorylation of IRS-1^{7A} in Fao cells. FIG. 3a—Fao cells were infected with Ad-Myc-IRS-1^{WT} or Ad-Myc-IRS-1^{7A} at 7×10⁷ PFU/ml. Following 48 hours, the cells were starved in serum-free RPMI medium for 16 h and were then treated with insulin for the indicated times. Cell extracts were prepared and samples (1 mg) were subjected to immunoprecipitation (IP) with anti-Myc antibodies. Immunocomplexes were resolved on 7.5% SDS-PAGE and immunoblotted with anti-pY antibodies. In parallel, samples (100 μg) of total cell extracts were resolved on 7.5% SDS-PAGE and immunoblotted with anti-Myc antibodies. FIG. 3b—The bands corresponding to anti-pY/total Myc-IRS-1 were quantified by densitometry and are presented as a bar graph. Results are of a representative experiment carried out in duplicate.

FIGS. 4a-c show the effect of insulin on activation of PKB, MAPK, and GSK-3β in Fao cells infected with IRS-1^{WT} or IRS-1^{7A}. Fao cells at 70% were infected with Ad-Myc-IRS-1^{WT} or Ad-Myc-IRS-1^{7A} at 6×10⁷ PFU/ml. Following 48 hours the cells were starved in serum-free medium for 16 hours and were further incubated with 100 nM insulin for the indicated time at 37° C. Cytosolic extracts were prepared; samples (100 μg) were resolved by means of 7.5% SDS-PAGE and were immunoblotted with the indicated antibodies. The results of four independent experiments were quantified and were normalized relative to the total cellular content of the protein under study (FIGS. 4a and 4c). Results of a representative experiment are shown in FIG. 4b.

FIGS. 5a-c show the effects of TPA and anisomycin on insulin-induced Tyr phosphorylation of IRS-1^{7A} in Fao cells. Fao cells were infected with Ad-Myc-IRS-1^{WT} or Ad-Myc-IRS-1^{7A} at 7×10⁷ PFU/ml. Following 48 hours the cells were deprived of serum for 16 h, and were then treated for 60 min with 200 nM TPA (FIG. 5a); 50 ng/ml of anisomycin (FIG. 5b) or 1 μg/ml of anisomycin (FIG. 5c). This was followed by treatment with 100 nM insulin for 2 min (FIG. 5a) or for the indicated times (FIGS. 5b-c). Cell extracts were prepared, and samples (1 mg) were subjected to immunoprecipitation (IP) with anti-Myc antibodies (FIG. 5a). Immunocomplexes were resolved on 7.5 SDS-PAGE and were immunoblotted with the indicated antibodies. Alternatively, total cell extracts (FIGS. 5b-c) were resolved on 7.5% SDS-PAGE and immunoblotted with the indicated antibodies. Results of a representative of four experiments are presented. The results of two independent experiments done in duplicates were quantified (FIG. 5a, right panel).

FIGS. 6a-b show the dose dependent effect of Palmitic acid on Insulin-induced Tyr phosphorylation of IRS-1 mutants in Fao cells. Fao cells were infected with Ad-Myc-IRS-1^{WT} or Ad-Myc-IRS-1^{7A} at 7×10⁷ PFU/ml. Following 24 hours the cells were deprived of serum for 12 h, and were then further incubated for 12 h with or without the indicated concentrations of palmitic acid in serum free medium prior to being stimulated with 100 nM insulin for the indicated times. Cell extracts (100 μg) were resolved on 7.5% SDS-PAGE and immunoblotted with anti-PY and anti-Myc antibodies. The results of two independent experiments done in duplicates were quantified.

FIGS. 7a-c show In vivo phosphorylation of PH/PTB-L in response to insulin. CHO-T cells were transiently transfected with pcDNA3-Myc-PH/PTB (1-1290 nt) or with an insert-free plasmid (control). Twenty-four hours post-transfection, the cells were deprived of serum for 16 h and were then treated with insulin for the indicated time periods. Cell extracts (100 μg) were resolved on 10% SDS-PAGE and immunoblotted with anti-pY or anti-Myc antibodies (FIG. 7a). In parallel, samples (100 μg, 40 μl) were incubated with 3 units (U) of calf intestine phosphatase (CIP) at 37° C. for 1 h. The samples were resolved on 10% SDS-PAGE and immunoblotted with anti-Myc antibodies (FIG. 7b). The results are of a representative of two experiments. FIG. 7c—CHO^{PH/PTB-L} cells were starved in serum-free F12 medium for 16 h. The cells were treated for 30 min with the indicated inhibitors prior to being treated with 100 nM insulin for 30 min. Cell extracts were prepared, samples were resolved on 10% SDS-PAGE and were immunoblotted with anti-Myc antibodies. Results are of a representative of three experiments.

FIGS. 8a-b show the effects of insulin on Serine phosphorylation of Myc-PH/PTB-L^{7A} CHO-T cells were transiently transfected with constructs encoding Myc-PH/PTB-L (1-1290 nt), either wild type (WT) or the 7A mutant (FIG. 8a). Alternatively, the cells were transfected or with the constructs encoding Myc-PH/PTB-L (1-1290 nt; 1-430 aa) or Myc-PH/PTB-S (1-1095 nt; 1-365 aa, FIG. 8b). Following 24 hours in culture, the cells were incubated in serum-free F12 medium for 16 ch prior to being treated with insulin for the indicated times. Cell extracts were resolved on 10% SDS-PAGE and immunoblotted with anti-Myc antibodies. Results are of a representative of three experiments.

FIGS. 9a-b show the effects of insulin and calyculin A on Ser phosphorylation of PH/PTB-L mutants. FIG. 9a—CHO-T cells were transiently transfected with constructs expressing either Myc-PH/PTB^{WT} or the following PH/PTB mutants: S336A, S407A, S408A, S407/8A and S3A (S336/407/408A). Following 24 hours the cells were deprived of serum for 16 h, and were treated with insulin for the indicated times. Cell extracts were resolved on 10% SDS-PAGE and immunoblotted with anti-Myc antibodies. The results are of a representative of two-three experiments. FIG. 9b—CHO-T cells were transiently transfected with pcDNA3-myc-PH/PTB^{WT} or pcDNA3-myc-PH/PTB^{S408A}. Following 24 hours the cells were starved for 16 h and were then treated either with 100 nM insulin for 30 min or with 25 nM calyculin A for 60 min. Cell extracts were resolved on 10% SDS-PAGE and were immunoblotted with anti-Myc antibodies. Results are of a representative of two experiments.

FIGS. 10a-b shows phosphorylation of S408 of IRS-1 in cultured Fao cells. Fao cells were infected with Adeno-myc-IRS-1 wild type (WT); the 7A mutant; or the S408A mutant at a titer of 4-6×10⁷ PFU/ml, so as to obtain comparable expression levels of the proteins under study (compare with FIGS. 11a-b). Following 48 hours, the cells were starved for 16 hours. The medium was removed and the cells were incubated with or without 100 nM insulin for the indicated times (FIG. 10a). Alternatively, the cells were incubated with insulin, SMase or calyculin A as indicated (FIG. 10b). Cytosolic extracts (100 μg) were resolved on 7.5% SDS-PAGE and immunoblotted with anti-phospho-Ser 408 antibody or anti-PY antibodies as indicated.

FIGS. 11a-c show the effects of TPA and anisomycin on insulin-stimulated Tyr phosphorylation of IRS-1^{WT}, IRS-1^{7A} and IRS-1^{408A}. Fao cells were infected with Adeno-myc-IRS-1 wild type (WT); the 7A mutant; or the S408A mutant at a titer of 4-6×10⁷ PFU/ml. Following 48 hours the cells were deprived of serum for 16 h, and were then treated for 60 min with 200 nM TPA (FIG. 11a), or 50 ng/ml anisomycin (FIG. 11b), followed by treatment with 100 nM insulin for the indicated times. Cell extracts were prepared, samples (1 mg) were subjected to immunoprecipitation (IP) with anti-Myc antibodies. Immunocomplexes were resolved on 7.5% SDS-PAGE and immunoblotted with anti-PY or anti IRS-1 antibodies. FIG. 11c shows quantification of results of two independent experiments done in duplicates.

FIGS. 12a-b are multiple alignment schemes depicting sequence conservation between amino acid sequences of IRS-1 and IRS-2 (FIG. 12a) as well as between amino acid sequences of IRS-1 and IRS-4 (FIG. 12b). Conserved serine residues of the present invention are highlighted (in bold).

### DESCRIPTION OF THE PREFERRED EMBODIMENTS

The present invention is of compositions for- and methods of diagnosing and treating insulin resistance. Specifically, the present invention can be used to diagnose and treat Type II diabetes.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Insulin resistance, which is commonly associated with prevalent type 2 diabetes, is a state in which target cells fail to respond to normal levels of circulating insulin. This lack of response, in turn, results in hyperinsulinemia to compensate for the resistance to insulin in the prediabetic state. Subsequently, hyperglycemia develops due to the failure of the pancreatic beta cells to produce and secrete enough insulin to compensate for the imbalance in glucose metabolism.

At the molecular level, insulin resistance may result from mutations or posttranslational modifications of the insulin receptor or any of its downstream targets, especially IRS-1, the insulin receptor signaling unit.

IRS-1 contains over 70 potential Ser/Thr phosphorylation sites with homologies to consensus phosphorylation sites for casein kinase II, PKB, PKC, MAP kinases, CDC2, cAMP- and cGMP-dependent protein kinase. Phosphorylation of Ser/Thr residues of IRS proteins has a dual function and serves either for a positive or for a negative modulation of insulin signal transduction.

Ser/Thr phosphorylation can induce the dissociation of IRS proteins from the insulin receptor (IR); hinder Tyr phosphorylation sites; release the IRS proteins from intracellular complexes that maintain them in close proximity to the receptor; induce IRS proteins degradation; or turn IRS proteins into inhibitors of the IRK. These multiple effects suggest that the Ser sites subjected to phosphorylation play a key role in regulating IRS-1 function. In fact inducers of insulin resistance such as free-fatty acids take advantage of this physiological shut-off mechanism and activate Ser/Thr kinases that phosphorylate the IRS-1 at the same inhibitory sites.

Accordingly, there remains a need for the identification of Ser/Thr phosphorylation sites in IRS proteins that are essential for the inhibition of insulin signaling leading to insulin resistance and type 2 diabetes.

While reducing the present invention to practice the present inventors uncovered serine (ser) phosphorylation sites which negatively regulate IRS-1 function and as such can be used to predict and diagnose insulin resistance and to design new therapeutic tools for treating and/or preventing insulin resistance.

As is illustrated hereinbelow and in the Examples section which follows, the present inventors showed that mutation of seven Ser residues, six located at putative PKC phosphorylation sites (RXXS) proximal to the PTB domain of IRS-1, renders the mutated IRS-1 protein resistant to the inhibitory effects of IRS-1 kinases stimulated either by insulin or by agents that induce insulin resistance. Utilizing truncated forms of mouse IRS-1 they were able to show that Ser408 (corresponding to Ser413 of human IRS-1) is a prime ‘inhibitory’ phosphorylation site, located within a region (aa 365-430) subjected to Ser phosphorylation in response to insulin or inducers of insulin resistance. These findings suggest that S408 and additional Ser sites among the mutated 7S, are targets for IRS-1 kinases that play a key negative regulatory role in IRS-1 function and insulin action.

Thus, according to one aspect of the present invention there is provided a method of treating insulin resistance in a subject.

As used herein the phrase “insulin resistance” refers to a state in which target cells fail to respond to normal levels of circulating insulin. At the molecular level insulin resistance relates to abnormally reduced signaling and metabolic outcome (e.g., glucose uptake, metabolism, or storage) through the insulin receptor pathway which involves signaling via an IRS protein, as further described hereinbelow. Insulin resistance is associated with numerous medical conditions (i.e., causes or is a consequence of) including, but not limited to, diabetes (e.g., Type 2 diabetes), obesity, hyperglycemia, hyperlipidemia, acute trauma, chronic infection and cardiovascular diseases (see 27, 33).

As used herein the term “treating” refers to preventing, alleviating or diminishing a symptom associated with insulin resistance. Preferably, treating cures, e.g., substantially eliminates, the symptoms associated with insulin resistance.

As used herein the term “subject” refers to an animal subject (e.g., mammal), preferably a human subject.

The method according to this aspect of the present invention comprises administering to the subject a therapeutically effective amount of an agent capable of down-regulating phosphorylation of an IRS protein at at least one serine residue corresponding to amino acid 270, 307, 330, 341, 412 and/or 413 of human IRS-1, thereby treating the insulin resistance in the subject.

As used herein the phrase “IRS protein” refers to homologues and orthologues of Insulin Receptor Substrate proteins. Examples include, but are not limited to, IRS-1 (GenBank Accession No. NP_{—}005535.1), IRS-2 (GenBank Accession No. NP_{—}003740.2) and IRS-4 (GenBank Accession No. NP_{—}003595).

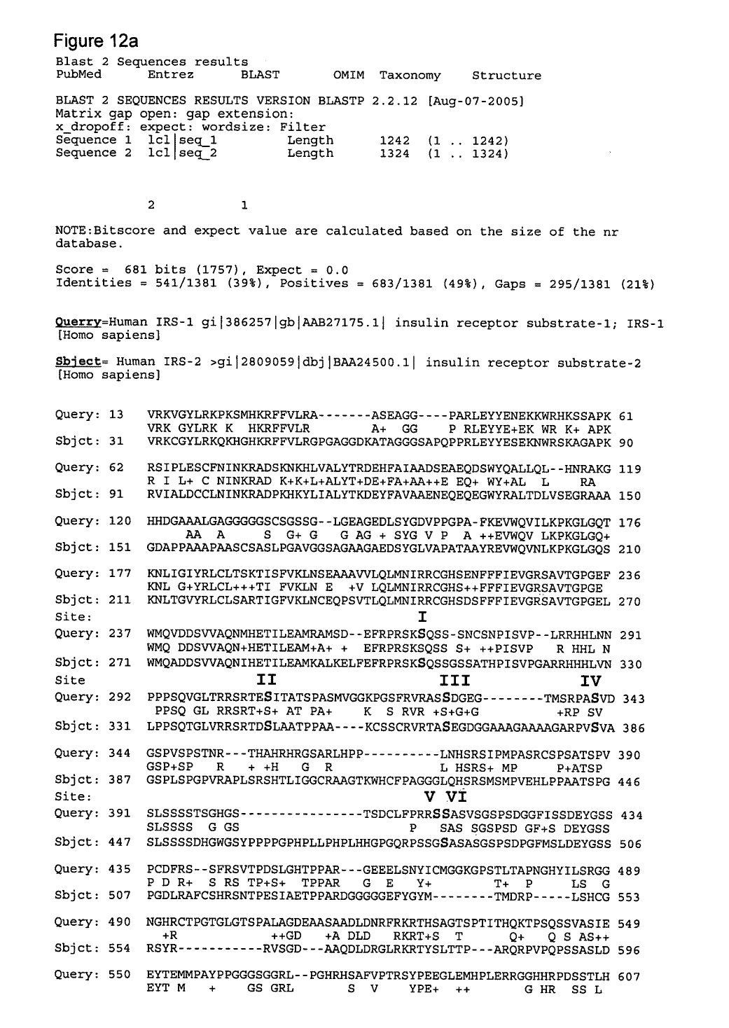

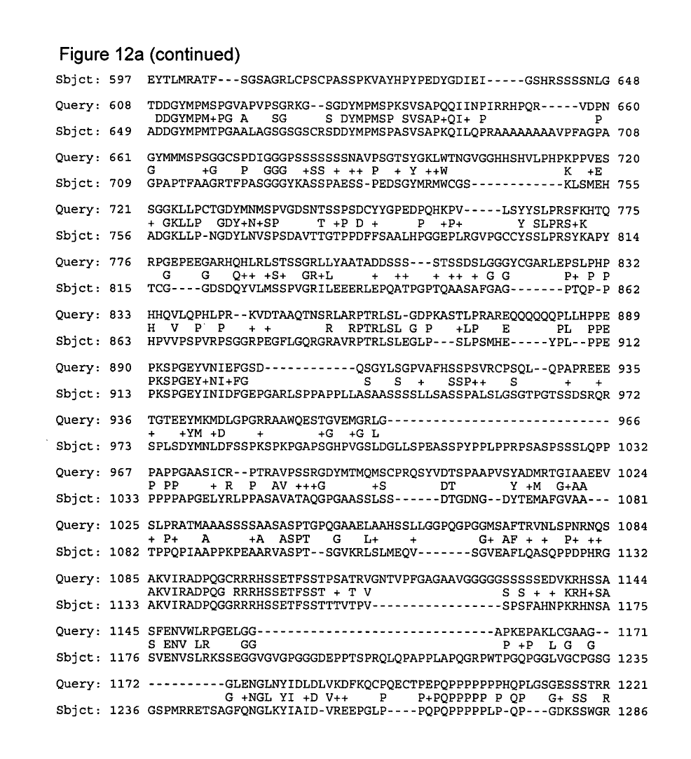

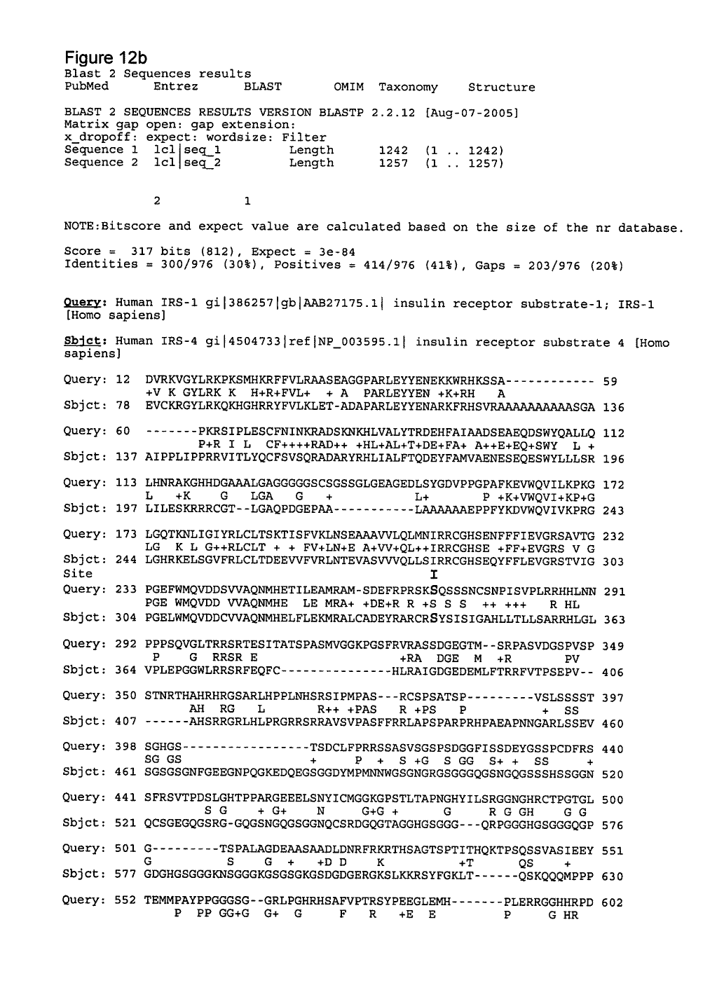

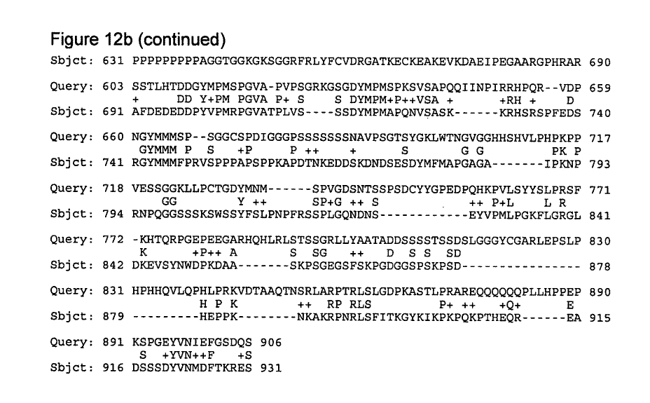

Table 1 below lists the IRS-1 Serine phosphorylation sites of the present invention and their homologous positions in mouse and human IRS-2 and IRS-4 (shown also in FIGS. 12a-b).

TABLE 1

| Mouse | Human | Mouse | Human | Human |
|---|---|---|---|---|
| IRS-1 | IRS-1 | IRS-2 | IRS-2 | IRS-4 |
| Ser265 | Ser270 | Ser303 | Ser306 | 342 |
| Ser302 | Ser307 | Ser343 | Ser346 | |
| Ser325 | Ser330 | Ser362 | Ser365 | |
| Ser336 | Ser341 | Ser381 | Ser384 | |
| Ser407 | Ser412 | — | — | |
| Ser408 | Ser413 | Ser 482 | Ser 485 | |

Various agents (e.g., protein agents, nucleic acid agents and small molecule chemical agents) can be used to down-regulate (i.e., reduce below normal IRS Ser phosphorylation levels in functional insulin responding cells) phosphorylation of the IRS protein at the above-described sites.

According to one preferred embodiment of this aspect of the present invention, the agent is a peptide agent which comprises an IRS amino acid sequence including at least one of the above-described serine residues (in a native non-phosphorylated form). Such a peptide agent preferably does not exceed 100 amino acids in length, more preferably does not exceed 50 amino acids in length, even more preferably does not exceed 20 amino acids in length, yet even more preferably does not exceed 10 amino acids in length.

Without being bound by theory, it is suggested that such a peptide agent will be phosphorylated by the native IRS phosphorylating protein (e.g., PKC, PKB and GSK3), thereby competing with the native IRS protein allowing the latter to maintain its active conformation. An example of such a peptide agent corresponds to amino acids 386-434 of mouse IRS-1 (see FIG. 12a). Other examples are provided in in SEQ ID NOs: 1-8.

The term “peptide” as used herein encompasses native peptides (either degradation products, synthetically synthesized peptides, or recombinant peptides), peptidomimetics (typically, synthetically synthesized peptides), and the peptide analogues peptoids and semipeptoids, and may have, for example, modifications rendering the peptides more stable while in a body or more capable of penetrating into cells. Such modifications include, but are not limited to: N-terminus modifications; C-terminus modifications; peptide bond modifications, including but not limited to CH₂—NH, CH₂—S, CH₂—S═O, O═C—NH, CH₂—O, CH₂—CH₂, S═C—NH, CH═CH, and CF═CH; backbone modifications; and residue modifications. Methods for preparing peptidomimetic compounds are well known in the art and are specified, for example, in Ramsden, C. A., ed. (1992), Quantitative Drug Design, Chapter 17.2, F. Choplin Pergamon Press, which is incorporated by reference as if fully set forth herein. Further details in this respect are provided hereinbelow.

Peptide bonds (—CO—NH—) within the peptide may be substituted, for example, by N-methylated bonds (—N(CH3)-CO—); ester bonds (—C(R)H—C—O—O—C(R)—N—); ketomethylene bonds (—CO—CH2-); α-aza bonds (—NH—N(R)—CO—), wherein R is any alkyl group, e.g., methyl; carba bonds (—CH2-NH—); hydroxyethylene bonds (—CH(OH)—CH2-); thioamide bonds (—CS—NH—); olefinic double bonds (—CH═CH—); retro amide bonds (—NH—CO—); and peptide derivatives (—N(R)—CH2-CO—), wherein R is the “normal” side chain, naturally presented on the carbon atom. These modifications can occur at any of the bonds along the peptide chain and even at several (2-3) at the same time.

Natural aromatic amino acids, Trp, Tyr, and Phe, may be substituted for synthetic non-natural acids such as, for instance, tetrahydroisoquinoline-3-carboxylic acid (TIC), naphthylelanine (Nol), ring-methylated derivatives of Phe, halogenated derivatives of Phe, and o-methyl-Tyr.

In addition to the above, the peptides of the present invention may also include one or more modified amino acids or one or more non-amino acid monomers (e.g., fatty acids, complex carbohydrates, etc.).

The term “amino acid” or “amino acids” is understood to include the 20 naturally occurring amino acids; those amino acids often modified post-translationally in vivo, including, for example, hydroxyproline, phosphoserine, and phosphothreonine; and other less common amino acids, including but not limited to 2-aminoadipic acid, hydroxylysine, isodesmosine, nor-valine, nor-leucine, and ornithine. Furthermore, the term “amino acid” includes both D- and L-amino acids.

Tables 2 and 3 below list naturally occurring amino acids (Table 2) and non-conventional or modified amino acids (Table 3) which can be used with the present invention.

TABLE 2

| Amino Acid | Three-Letter Abbreviation | One-letter Symbol |
|---|---|---|
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid | Xaa | X |
| as above | | |

TABLE 3

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The peptides of the present invention are preferably utilized in a linear form, although it will be appreciated that in cases where cyclization does not severely interfere with peptide characteristics, cyclic forms of the peptide can also be utilized.

It should be noted that since IRS is an intracellular protein, the peptide agent (and other agents of the present invention, described hereinbelow) is preferably designed to penetrate through the plasma membrane. Thus, for example, the peptide agent may be designed to include hydrophobic moieties either as part of the peptide backbone, or attached thereto (e.g., such as by attaching a fatty acid moiety thereto). Such peptide modifications may be effected as long as peptide activity is not compromised. Methods of testing peptide activity are described in length in the Examples section which follows.

The peptides of the present invention may be synthesized by any techniques that are known to those skilled in the art of peptide synthesis. For solid phase peptide synthesis, a summary of the many techniques may be found in: Stewart, J. M. and Young, J. D. (1963), “Solid Phase Peptide Synthesis,” W. H. Freeman Co. (San Francisco); and Meienhofer, J (1973). “Hormonal Proteins and Peptides,” vol. 2, p. 46, Academic Press (New York). For a review of classical solution synthesis, see Schroder, G. and Lupke, K. (1965). The Peptides, vol. 1, Academic Press (New York).

In general, peptide synthesis methods comprise the sequential addition of one or more amino acids or suitably protected amino acids to a growing peptide chain. Normally, either the amino or the carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth; traditionally this process is accompanied by wash steps as well. After all of the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide, and so forth.

Further description of peptide synthesis is disclosed in U.S. Pat. No. 6,472,505. A preferred method of preparing the peptide compounds of the present invention involves solid-phase peptide synthesis, utilizing a solid support. Large-scale peptide synthesis is described by Andersson Biopolymers 2000, 55(3), 227-50.

According to another embodiment of this aspect of the present invention, the agent is an isolated polynucleotide (i.e., nucleic acid agent) which comprises a nucleic acid sequence encoding an IRS protein comprising a mutation (e.g., deletion, nucleic acid substitution) in at least one of the above described serine residue.

As used herein the phrase “an isolated polynucleotide” refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

According to one preferred embodiment of this aspect of the present invention, the isolated polynucleotide agent encompasses substitution mutations in all the above described serine residues (e.g., corresponds to the mouse 7A IRS-1 mutant) or in a portion thereof (e.g., the S408A IRS-1 mutant described in length in the Examples section which follows). Alternatively, the isolated polynucleotide agent of the present invention is a deletion mutant which comprises a deletion in at least one of the above-described serine residues. An example for such a nucleic acid sequence deletion is the Δ386-434 of mouse IRS-1. Such an IRS-1 mutant is resistant to protein degradation and as such may improve insulin signaling therethrough (data not shown).

Nucleic acid agents of the present invention are preferably ligated into a nucleic acid expression construct, which allows expression thereof in mammalian cells. It will be appreciated that the nucleic acid construct can be administered to the subject employing any suitable mode of administration, described hereinbelow (i.e., in-vivo gene therapy). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the subject (i.e., ex-vivo gene therapy).

To enable cellular expression of the polynucleotides of the present invention, the nucleic acid construct of the present invention further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277] or the phosphoglycerate kinase 1 promoter; in particular pancreas-specific promoters [e.g., Edlunch et al. (1985) Science 230:912-916]. The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, pox virus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

According to yet another embodiment of this aspect of the present invention the agent is a chemical agent (i.e., small molecule or large molecule) which inhibitos phosphorylation of IRS on the above-described sites. An example for such an inhibitor is a PKC inhibitor. For further details see the Examples section which follows. Such inhibitors are available from Sigma-Aldrich (e.g., GF109). Other PKC inhibitors include, but are not limited to, Calphostin C, Chelerythrine chloride, Go 6976, Ro-32-0432, Ro-31-7549, Ro-31-8220, Ro-31-8425, Ro-32-0432, and Rottlerin (commercially available from Calbiochem).

Based on the above findings, the present invention further provides a method of identifying other agents which are suitable for the treatment of insulin resistance. The method is effected by contacting a biological sample comprising an IRS protein phosphorylated on at least one of the above-described serine residue with a plurality of agents; and identifying at least one agent of the plurality of agents being capable of dephosphorylating the at least one serine residue, thereby identifying agents suitable for treating insulin resistance.

As used herein the term biological sample, refers to a cellular sample which expresses an IRS protein. Examples of such biological samples include, but are not limited to, serum, spinal fluid, lymph fluid, skin samples, respiratory, intestinal, and genitourinary tracts, pancreatic samples liver samples, saliva, milk, blood cells, tumors, tissues (e.g., adipose), organs, and also samples of in vivo cell culture constituents (e.g., skeletal muscle primary culture, 3).

The sample may be treated with inducers of insulin resistance or taken from insulin resistant patients as further described hereinbelow.

Methods of detecting the phosphorylation status (i.e., phosphorylated/unphosphorylated) of an IRS protein are well known in the art. For example, by using anti phospho-serine antibodies (e.g., anti Ser 408, described in the Examples section which follows), as further described hereinbelow.

The agent(s) of the present invention can be administered to the subject per se, or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the agent accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

To improve therapeutic efficacy, agents of the present invention may be administered along with other anti-insulin resistance drugs which are well known in the art (i.e., combination therapy). A number of medicaments for the treatment and prevention of insulin resistance are available for use in accordance with the present invention. The most widely used agents are the thiazolidinedione (TZD) insulin-sensitizing agents. TZDs are a class of compounds that improve insulin action in vivo which have also been introduced as therapeutic agents for the treatment of type 2 diabetes. Since their introduction, it has been discovered that TZDs act as agonists of the peroxisome proliferator-activated receptor gamma (PPAR-gamma) nuclear receptor. TZD's have been shown to improve insulin sensitivity in human subjects, including also in obese subjects [Nolan et al, (1994) N. Eng. J. Med. 331: 1188-1193]. Examples of suitable TZDs include troglitazone [Eli Lilly, Indianapolis, U.S.A.], rosiglitazone, and pioglitazone.

In addition to therapeutic advances pioneered by the present invention, the present invention further provides methods of diagnosing insulin resistance.

Thus, according to another aspect of the present invention there is provided a method of diagnosing insulin resistance in a subject.

As used herein the term “diagnosing” refers to classifying a disease or a symptom as associated with insulin resistance, determining a severity of the disease, monitoring disease progression, assessing predisposition (i.e., the risk of developing the disease), forecasting an outcome of a disease and/or prospects of recovery.

The method comprising detecting in a biological sample of the subject, presence, absence or level of an IRS protein phosphorylated on at least one serine residue corresponding to amino acid 341, 412 and/or 413 of human IRS-1, wherein the presence or level of the phosphorylated IRS protein is indicative of the insulin resistance in the subject.

Methods of obtaining biological samples from subjects are well known in the art. Examples include, but are not limited to, fine needle biopsy, needle biopsy, core needle biopsy and surgical biopsy (e.g., liver biopsy).

Regardless of the procedure employed, once a biopsy is obtained the level of the phosphorylation at the sites of IRS protein of the present invention can be determined and a diagnosis can thus be made.

As used herein the term “level” refers to the number of the above-serine sites (e.g., 341, 412 and/or 413 of human IRS-1) which are phosphorylated per a single IRS molecule and/or the concentration of phosphorylated IRS molecules.

Detecting the presence, absence or level of phosphorylated IRS protein may be effected using biochemical methods (e.g., in-vitro kinase assay and gel retardation assay) and immunological methods. The latter may be effected using an antibody or an antibody fragment comprising an antigen recognition domain capable of specifically binding an IRS protein phosphorylated on at least one of the above serine residues but does not bind the IRS protein when not phosphorylated on this respective position.

Methods of generating such antibodies are well known in the art and described at length hereinbelow and in the Examples section which follows.

As used herein, the term “antibody” refers to a substantially intact antibody molecule.

As used herein, the phrase “antibody fragment” refers to a functional fragment of an antibody that is capable of binding to an antigen.

Suitable antibody fragments for practicing the present invention include, inter alia, a complementarity-determining region (CDR) of an immunoglobulin light chain (referred to herein as “light chain”), a CDR of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single-chain Fv, an Fab, an Fab′, and an F(ab′)2.

Functional antibody fragments comprising whole or essentially whole variable regions of both light and heavy chains are defined as follows:

(i) Fv, defined as a genetically engineered fragment consisting of the variable region of the light chain and the variable region of the heavy chain expressed as two chains;

(ii) single-chain Fv (“scFv”), a genetically engineered single-chain molecule including the variable region of the light chain and the variable region of the heavy chain, linked by a suitable polypeptide linker.

(iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof;

(iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and

(v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds).

Methods of generating monoclonal and polyclonal antibodies are well known in the art. Antibodies may be generated via any one of several known methods, which may employ induction of in vivo production of antibody molecules, screening of immunoglobulin libraries (Orlandi, R. et al. (1989). Cloning immunoglobulin variable domains for expression by the polymerase chain reaction. Proc Natl Acad Sci USA 86, 3833-3837; and Winter, G. and Milstein, C. (1991). Man-made antibodies. Nature 349, 293-299), or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler, G. and Milstein, C. (1975). Continuous cultures of fused cells secreting antibody of predefined specificity. Nature 256, 495-497; Kozbor, D. et al. (1985). Specific immunoglobulin production and enhanced tumorigenicity following ascites growth of human hybridomas. J Immunol Methods 81, 31-42; Cote R J. et al. (1983). Generation of human monoclonal antibodies reactive with cellular antigens. Proc Natl Acad Sci USA 80, 2026-2030; and Cole, S. P. et al. (1984). Human monoclonal antibodies. Mol Cell Biol 62, 109-120).

In cases where target antigens are too small to elicit an adequate immunogenic response when generating antibodies in vivo, such antigens (referred to as “haptens”) can be coupled to antigenically neutral carriers such as keyhole limpet hemocyanin (KLH) or serum albumin (e.g., bovine serum albumin (BSA)) carriers (see, for example, US. Pat. Nos. 5,189,178 and 5,239,078). Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill., USA. The resulting immunogenic complex can then be injected into suitable mammalian subjects such as mice, rabbits, and others. Suitable protocols involve repeated injection of the immunogen in the presence of adjuvants according to a schedule designed to boost production of antibodies in the serum. The titers of the immune serum can readily be measured using immunoassay procedures which are well known in the art.

The antisera obtained can be used directly or monoclonal antibodies may be obtained, as described hereinabove.

Antibody fragments may be obtained using methods well known in the art. (See, for example, Harlow, E. and Lane, D. (1988). Antibodies: A Laboratory Manual, Cold Spring Harbor Laboratory, New York.) For example, antibody fragments according to the present invention can be prepared by proteolytic hydrolysis of the antibody or by expression in E. coli or mammalian cells (e.g., Chinese hamster ovary (CHO) cell culture or other protein expression systems) of DNA encoding the fragment.

Alternatively, antibody fragments can be obtained by pepsin or papain digestion of whole antibodies by conventional methods. As described hereinabove, an (Fab′)2 antibody fragments can be produced by enzymatic cleavage of antibodies with pepsin to provide a 5S fragment. This fragment can be further cleaved using a thiol reducing agent, and optionally a blocking group for the sulfhydryl groups resulting from cleavage of disulfide linkages, to produce 3.5S Fab′ monovalent fragments. Alternatively, enzymatic cleavage using pepsin produces two monovalent Fab′ fragments and an Fc fragment directly. Ample guidance for practicing such methods is provided in the literature of the art (for example, refer to: U.S. Pat. Nos. 4,036,945 and 4,331,647; and Porter, R. R. (1959). The hydrolysis of rabbit γ-globulin and antibodies with crystalline papain. Biochem J 73, 119-126). Other methods of cleaving antibodies, such as separation of heavy chains to form monovalent light-heavy chain fragments, further cleavage of fragments, or other enzymatic, chemical, or genetic techniques may also be used, so long as the fragments retain the ability to bind to the antigen that is recognized by the intact antibody.

As described hereinabove, an Fv is composed of paired heavy chain variable and light chain variable domains. This association may be noncovalent (see, for example, Inbar, D. et al. (1972). Localization of antibody-combining sites within the variable portions of heavy and light chains. Proc Natl Acad Sci USA 69, 2659-2662). Alternatively, as described hereinabove, the variable domains may be linked to generate a single-chain Fv by an intermolecular disulfide bond, or alternately such chains may be cross-linked by chemicals such as glutaraldehyde.

Preferably, the Fv is a single-chain Fv. Single-chain Fvs are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains. Ample guidance for producing single-chain Fvs is provided in the literature of the art (see, e.g.: Whitlow, M. and Filpula, D. (1991). Single-chain Fv proteins and their fusion proteins. METHODS: A Companion to Methods in Enzymology 2(2), 97-105; Bird, R. E. et al. (1988). Single-chain antigen-binding proteins. Science 242, 423-426; Pack, P. et al. (1993). Improved bivalent miniantibodies, with identical avidity as whole antibodies, produced by high cell density fermentation of Escherichia coli. Biotechnology (N.Y.) 11(11), 1271-1277; and U.S. Pat. No. 4,946,778).

Isolated complementarity-determining region peptides can be obtained by constructing genes encoding the CDR of an antibody of interest. Such genes may be prepared, for example, by RT-PCR of the mRNA of an antibody-producing cell. Ample guidance for practicing such methods is provided in the literature of the art (e.g., Larrick, J. W. and Fry, K. E. (1991). PCR Amplification of Antibody Genes. METHODS: A Companion to Methods in Enzymology 2(2), 106-110).

It will be appreciated that for human therapy or diagnostics, humanized antibodies are preferably used. Humanized forms of non-human (e.g., murine) antibodies are genetically engineered chimeric antibodies or antibody fragments having (preferably minimal) portions derived from non-human antibodies. Humanized antibodies include antibodies in which the CDRs of a human antibody (recipient antibody) are replaced by residues from a CDR of a non-human species (donor antibody), such as mouse, rat, or rabbit, having the desired functionality. In some instances, the Fv framework residues of the human antibody are replaced by corresponding non-human residues. Humanized antibodies may also comprise residues found neither in the recipient antibody nor in the imported CDR or framework sequences. In general, the humanized antibody will comprise substantially all of at least one, and typically two, variable domains, in which all or substantially all of the CDRs correspond to those of a non-human antibody and all or substantially all of the framework regions correspond to those of a relevant human consensus sequence. Humanized antibodies optimally also include at least a portion of an antibody constant region, such as an Fc region, typically derived from a human antibody (see, for example: Jones, P. T. et al. (1986). Replacing the complementarity-determining regions in a human antibody with those from a mouse. Nature 321, 522-525; Riechmann, L. et al. (1988). Reshaping human antibodies for therapy. Nature 332, 323-327; Presta, L. G. (1992b). Curr Opin Struct Biol 2, 593-596; and Presta, L. G. (1992a). Antibody engineering. Curr Opin Biotechnol 3(4), 394-398).

Methods for humanizing non-human antibodies are well known in the art. Generally, a humanized antibody has one or more amino acid residues introduced into it from a source which is non-human. These non-human amino acid residues are often referred to as imported residues, which are typically taken from an imported variable domain. Humanization can be performed essentially as described (see, for example: Jones et al. (1986); Riechmann et al. (1988); Verhoeyen, M. et al. (1988). Reshaping human antibodies: grafting an antilysozyme activity. Science 239, 1534-1536; and U.S. Pat. No. 4,816,567), by substituting human CDRs with corresponding rodent CDRs. Accordingly, humanized antibodies are chimeric antibodies, wherein substantially less than an intact human variable domain has been substituted by the corresponding sequence from a non-human species. In practice, humanized antibodies may be typically human antibodies in which some CDR residues and possibly some framework residues are substituted by residues from analogous sites in rodent antibodies.

Human antibodies can also be produced using various additional techniques known in the art, including phage-display libraries (Hoogenboom, H. R. and Winter, G. (1991). By-passing immunisation. Human antibodies from synthetic repertoires of germline VH gene segments rearranged in vitro. J Mol Biol 227, 381-388; Marks, J. D. et al. (1991). By-passing immunization. Human antibodies from V-gene libraries displayed on phage. J Mol Biol 222, 581-597; Cole et al. (1985), Monoclonal Antibodies and Cancer Therapy, Alan R. Liss, Inc., pp. 77-96; and Boerner, P. et al. (1991). Production of antigen-specific human monoclonal antibodies from in vitro-primed human splenocytes. J Immunol 147, 86-95). Humanized antibodies can also be created by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807; 5,545,806; 5,569,825; 5,625,126; 5,633,425; and 5,661,016; Marks, J. D. et al. (1992). By-passing immunization: building high affinity human antibodies by chain shuffling. Biotechnology (N.Y.) 10(7), 779-783; Lonberg et al., 1994. Nature 368:856-859; Morrison, S. L. (1994). News and View: Success in Specification. Nature 368, 812-813; Fishwild, D. M. et al. (1996). High-avidity human IgG kappa monoclonal antibodies from a novel strain of minilocus transgenic mice. Nat Biotechnol 14, 845-851; Neuberger, M. (1996). Generating high-avidity human Mabs in mice. Nat Biotechnol 14, 826; and Lonberg, N. and Huszar, D. (1995). Human antibodies from transgenic mice. Int Rev Immunol 13, 65-93).

After antibodies have been obtained, they may be qualified for activity, for example via enzyme-linked immunosorbent assay. For example, the antibodies may be screened against the phospho and non-phospho peptide library by ELISA to ensure specificity for the desired antigen and for reactivity only with the phosphorylated form of the antigen. Peptide competition assays may be carried out to confirm lack of reactivity with other phosphoepitopes. The antibodies may also be tested by Western blotting against cell preparations containing IRS proteins, e.g. cell lines over-expressing IRS-1 and/or IRS-2, to confirm reactivity with the desired phosphorylated target. Specificity against the desired phosphorylated epitopes may also be examined by construction IRS mutants lacking phosphorylatable residues at positions outside the desired epitope known to be phosphorylated, or by mutating the desired phospho-epitope and confirming lack of reactivity.

In any case once antibodies are obtained they are contacted with the biological sample under conditions which allow for immuno-complex formation. Such conditions are described in the Examples section which follows. Immuno-complex formation may be qualified and/or quantified using various immunological assays which are well known in the art (e.g., ELISA, RIA, Western blot, Immunohistochemistry).

It will be appreciated that the present invention also envisages techniques for the in-vivo detection of insulin resistance using the reagents of the present invention. For example, the antibody can be labeled with a radioactive marker whose presence and location in a subject can be detected by standard imaging techniques.

Diagnostic kits for carrying out the methods disclosed above are also provided by the invention. Such kits comprise at least one detectable reagent for detecting phosphorylation of an IRS protein at the above Ser sites. In a preferred embodiment, the reagent is an anti phosphor Ser antibody as described herein. In one embodiment, the invention provides a kit for the detection of phosphorylated IRS-1 (e.g., Human IRS-1 Ser413) in a biological sample comprising (a) the antibody (b) optionally at least one secondary antibody conjugated to a detectable group (c) a solid phase for attaching multiple biological samples. The reagents may also include ancillary agents such as buffering agents and protein stabilizing agents, e.g., polysaccharides and the like. The diagnostic kit may further include, instructions for using the kit and where necessary, other members of the signal-producing system of which system the detectable group is a member (e.g., enzyme substrates), agents for reducing background interference in a test, control reagents, apparatus for conducting a test, and the like.

The diagnostic methods of the present invention may be effected in conjunction to blood evaluation (e.g., glucose levels) or surgical surveillance procedures. Such a diagnostic assay may be employed to identify insulin resistant patients. Such a selection of patients would be useful in the clinical evaluation of efficacy of currently available therapeutics as well as in the future prescription of future drugs to patients. Alternatively, the methods are applicable where a subject has been previously diagnosed as having a disease involving altered insulin signaling, such as type 2 diabetes or obesity, and possibly has already undergone treatment for the disease, and the method is employed to monitor the progression of the disease involving phosphorylation at the above sites, or the treatment thereof.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Experimental Procedures

Materials—Human insulin, wheat-germ agglutinin (WGA) coupled to agarose beads, glutathione-Agarose beads, protease inhibitors cocktail, protein A-Sepharose CL-4B, goat-anti-mouse antibodies coupled to Agarose beads, wortmannin and phorbol 12-myristate 13-acetate (TPA) were purchased from Sigma. Lipofectamin and OptiMem were from GIBCO-BRL (Grand Island N.Y.). Alkaline phosphatase was from Boehringer Mannheim (GmbH, Germany). T4 ligase, Gel-Extraction kit and pGEM-T were purchased from Promega. Tri-Reagent was purchased from Molecular Research Center, INC. SeaPlaque-Agarose was purchased from Bio Whittaker Molecular Applications (Rockland, Me., USA). Anisomycin, Palmitic acid, Calyculin A and SMase were purchased from Sigma Chemicals Co. (St. Louis, Mo.). Rapamycin, PD98059 and SB203580 were from Calbiochem (La Jolla, Calif.). Jet-PEI was purchased from Poly Transfection.

Antibodies—Monoclonal p-Tyr (PY-20) and polyclonal IRβ antibodies were obtained from Transduction Labs (Lexington, Ky.). Polyclonal IRS-1 antibodies were prepared as described (11). Polyclonal Myc antibodies were obtained from Santa Cruz Biotechnology, Inc. (Santa Cruz, Calif.). Polyclonal antibodies towards P-Ser307 of IRS-1 were from BioSource Inc. (Camarillo, Calif.). Rabbit polyclonal serum directed against phosphorylated Ser408 was generated using a synthetic peptide designed to contain phosphorylated Ser408 and surrounding amino acids CLFPRRSS(-P0₄)ASVSG, with additional Cys residue at the N-terminal end of the peptide (SEQ ID NO. 8). Peptide synthesis was effected at the peptide Synthesis facility of the Weizmann Institute (Rehovot, Israel) according to standard procedures.

Plasmids construction—i. Myc-tagged-IRS-1 The cDNA, encoding for the mouse IRS-1 in the pCNA-3 expression plasmid (35) was digested with Hind III and BspE-1. This deleted a 9 by fragment from the 5′-IRS-1 cDNA that was replaced by a double stranded synthetic oligonucleotide encoding the Myc tag and the missing amino-acids of IRS-1 (bold) MEQKLISEEDLNMASP (SEQ ID NO. 9). ii. Myc-tagged truncated IRS-1 (aa 1-430, PH/PTB-L) Myc-tagged mouse IRS-1 cDNA (N-terminus, 1-1290 nt), encoding for the first 430 aa of IRS-1, was PCR-amplified with the following primers. 5′-CAG GAT CCG CAT ATG GAA CAA AAG CTC-3′ (sense) (SEQ ID NO. 10); 5′-GAG AAT TCA TCA GGG ACT AGA ACC ATA-3′ (anti-sense) (SEQ ID NO. 11), using as a template the pcDNA3-Myc-IRS-1 plasmid. The PCR product was ligated into a pGEM-T. The insert was excised from pGEM-T with BamH I and EcoR I (italic), and then was ligated into pcDNA3 at the same restriction sites to generate pcDNA3-Myc-IRS-1^{PH/PTB-L}. iii. Myc-tagged truncated IRS-1 (aa 1-365, PH/PTB-S) Myc-tagged mouse IRS-1 cDNA (N-terminus, 1-1095 nt), encoding for the first 365 aa of IRS-1, was PCR-amplified with the following primers: 5′ AAGCTTAAGATATCGATCATATG-3′ (sense) (SEQ ID NO. 12); 5′ TTAGTTGAGTGGGGGGTGCAGCCT 3′ (anti sense) (SEQ ID NO. 13) using as a template the pcDNA3-Myc-IRS-1. The PCR product was ligated into a pGEM-T. The insert was excised from pGEM-T with EcoRV (italic) and NotI (within the pGEM-T construct), and was ligated into pcDNA3 at the same restriction sites.

Generation of IRS-1 mutants Site directed mutagenesis was performed with the following primers using a QuickChange Site-directed mutagenesis kit (Stratagene) according to the manufacturer's instructions. pcDNA3-IRS-1 served as a template. The mutated nucleotides are indicated in bold, mutated amino acid codes are in italics. The restriction sites introduced are underlined. The mutations were verified by restriction mapping and sequencing.

i. S336A 5′-C ATG TCC CGT CCA GCT GCA GTG GAT GGC AG-3′ (sense) (SEQ ID NO. 14); 5′-CT GCC ATC CAC TGC AGC TGG ACG GGA CAT G-3′ (antisense) (SEQ ID NO. 15). Pst I site introduced.

ii. S407A 5′-C TTC CCG AGG CGC GCT AGC GCT TCC GTG TCC GG-3′ (sense) (SEQ ID NO. 16); 5′-CC GGA CAC GGA AGC GCT AGC GCG CCT CGG GAA G-3′ (antisense) (SEQ ID NO. 17). Nhe I/Eco47 III sites introduced

iii. S408A 5′-C TTC CCG AGG CGC TCA GCT GCT TCC GTG TCC GG-3′ (sense) (SEQ ID NO. 18); 5′-CC GGA CAC GGA AGC AGC TGA GCG CCT CGG GAA G-3′ (antisense) (SEQ ID NO. 19). Pvu II site introduced

iv. S407/408A 5′-C TTC CCG A G̅G̅ C̅G̅C̅ G̅ C̅T̅ G̅C̅A̅ GCT TCC GTG TCC GG-3′ (sense) (SEQ ID NO. 20); 5′-CC GGA CAC GGA AGC TGC AGC GCG CCT CGG GAA G-3′ (antisense) (SEQ ID NO. 21). Pst I site introduced

v. IRS-1^{7A} The IRS-1^{7A} was generated on the basis of IRS-1^{4A}, in which four Ser residues S265, S302, S325, 5358 were mutated to Ala as described (26). The additional three Ser sites, S336, S407 and S408 were mutated into Ala sequentially using the above two sets of overlapping primers.

Transient and stable Transfections of CHO-T cells—Chinese Hamster Ovary cells, which overexpress the insulin receptor (CHO-T cells) (26), were transiently transfected with the pcDNA3-Myc-IRS-1^{PH/PTB-L} or pcDNA3-Myc-IRS-1^{PH/PTB-S} plasmids using Lipofectamin according to the protocol provided by Life Technologies. The transfected cells were cultured in F12 medium supplemented with 10% FCS. After 24 h, the cells were starved for 16h, then the indicated stimuli were applied. To generate stable clones, CHO-T cells were co-transfected with the pcDNA3-IRS-1^{wt} or the pcDNA3-IRS-1^{7A} plasmids encoding for the wild-type or the 7A mutant of IRS-1, respectively, together with pBabe-puro encoding for puromycin-resistance. After 24 h, the transfected cells were subjected to selection with 10 μg/ml puromycin. Stable clones expressing proper amounts of the target protein were selected and further propagated in a medium containing 10 μg/ml puromycin.

Generation of Adenoviral-based IRS-1 constructs—Adenoviruses harboring the genes of interest were generated according to the protocol provided with the AdEasy™ vector system (Quantum) (13). In brief, the target cDNA, encoding Myc-tagged mouse IRS-1 (wild-type, 7A mutant, and the S408A mutant) were ligated into the shuttle plasmid pAdTrack-CMV at an EcoR V restriction site. The correct orientation was confirmed by restriction mapping with Xho I or Kpn I, which yielded a ˜200 bp or ˜300 bp fragment, respectively. This plasmid also contains a GFP cassette whose expression is driven by an independent promoter, which serves as a tracing marker. The pAdTrack-CMV carrying the above target genes was co-transformed with pAdEasy-1, containing the adenovirus genome, into E. Coli strain, BJ5183, where homologous recombination took place. Positive colonies were identified by restriction analysis. The recombinant pAdEasy-1-IRS-1 plasmid (WT, 7A or S408A) were linearized with Pac I and were transfected into 293 cells using the Jet-PEI transfection reagent. Viruses, amplified in the 293 cells according the manufacturer's instructions, were stored at −80° C. at a viral titer of ˜1×10¹⁰ PFU/ml.

Infection of Fao cells with adenovirus—Fao cells were grown in RPMI medium containing 10% FCS. At 70% confluence cells were infected with adenoviruses harboring the gene of interest at a titer of 7 10⁷ PFU/ml. After incubation for 2 h hours at 37° C., the virus-containing medium was diluted 1:5 in fresh RPMI medium. After 24 h the medium was replaced with a fresh RPMI medium containing 10% FCS. 48 h post-infection the Fao cells were starved in serum-free RPMI for 16 h and were then subjected to the indicated treatments as described below.

Preparation of FFA solution—Preparation of solutions containing FFA complexed to BSA was carried out essentially as described (38). Briefly, a 100 mM Palmitic acid stock solution was prepared in 0.1 M NaOH at 70° C. The appropriate amount was then complexed with FFA-free 10% BSA solution at 55° C. for 30 min. The FFA/BSA complex was cooled down to room temperature and sterile filtered prior to its addition to the cell culture.

Treatment of cells and preparation of extracts—Virally-infected Fao cells were grown as described above. Nave Rat hepatoma (Fao) cells or CHO cells were grown in RPMI medium supplemented with 10% fetal calf serum as described (22). At 70-80% confluence, cells were deprived of serum for 16 h prior to each experiment, and were then incubated with inducers of insulin resistance and/or insulin. Treated cells were washed 3 times with ice-cold PBS, and were harvested in buffer B (25 mM Tris-HCl, 2 mM sodium orthovanadate, 0.5 mM EGTA, 10 mM NaF, 10 mM sodium pyrophosphate, 80 mM β-glycerophosphate, 25 mM NaCl, 1% Triton X-100 and protease inhibitor cocktail 1:1000, pH 7.4). Cell extracts were centrifuged at 12,000×g for 20 min at 4° C. and the supernatants were collected. Samples of 50-150 μg were mixed with 5× Laemmli sample buffer (17), boiled, and were resolved by SDS-PAGE under reducing conditions. The proteins were transferred into nitrocellulose membrane for Western blot with the indicated antibodies.

Immunoprecipitation—Protein A Sepharose beads (10-20 μl packed beads/point) or goat-anti-mouse antibodies, coupled to Agarose beads (10 μl packed beads/point) were washed three times with ice-cold 0.1M Tris-HCl (PH 8.0) and were incubated with the indicated antibodies in 0.1M Tris-HCl (PH 8.0) for 1 h at 4° C. 12,000×g supernatants of cell extracts in buffer B, containing 0.5-1.5 mg protein, were incubated for 2 h at 4° C. with the immobilized antibodies. Immunocomplexes were washed twice with buffer B, twice with ice-cold PBS and were mixed with 5× Laemmli sample buffer (17). The samples were boiled and then were resolved by SDS-PAGE under reducing conditions.

Assay of IR-IRS-1 complex formation—Fao cells at 80% confluence were harvested in buffer IR (50 mM Tris-HCl, 1% Nonidet P-40, 0.25% sodium deoxycholate, 150 mM NaCl, 1 mM EGTA, 1 mM sodium orthovanadate, 1 mM NaF, and protease inhibitor cocktail 1:1000, pH 7.4). The cell extracts were centrifuged at 12,000×g for 15 min at 4° C., and the supernatants were collected. Aliquots (1.0-1.5 mg) were incubated with 20 μl packed WGA-Agarose beads for 1 h at 4° C. The immobilized IR was washed twice with buffer IR and three times with buffer A (25 mM Tris-HCl, 2 mM sodium orthovanadate, 0.5 mM EGTA, 10 mM NaF, 10 mM sodium pyrophosphate, 80 mM β-glycerophosphate, 25 mM NaCl and protease inhibitor cocktail 1:1000, pH 7.4). 20 μl of the IR-WGA-coupled beads, were then incubated at 4° C. with 12,000×g supernatants of cytosolic extracts (1.0-1.5 mg), made in buffer A, derived from Fao cells treated with insulin for the indicated times. The beads were washed twice with buffer A and twice with ice-cold PBS, and then were boiled in Laemmli “sample buffer” (17). Samples were resolved by means of SDS-PAGE and were immunoblotted with IRS-1 antibodies.

In vitro Tyr kinase assay—Stable cell lines of CHO-T^{WT} and CHO-T^{7A} were grown in 90 mm plates in F12 medium supplemented with 10% FCS and 10 μg/ml puromycin. Cells were washed 3 times with PBS, and were harvested in 100 μl buffer A containing 0.5% NP40. Cell extracts were centrifuged at 12,000×g for 20 min at 4° C., and the supernatants were collected. Aliquots (90 μl, ˜1 mg) were incubated with 10⁻⁶ M insulin for 10 min at 22° C., then 90 μl of 2× buffer E (50 mM Hepes, 10 mM Mg acetate, 4 mM Mn acetate, 1 mM ATP, and protease inhibitor cocktail 1:1000, pH 7.4) were added and the incubation was continued for another 15 min at room temperature with vigorous shaking. The reaction was stopped by adding 200 μl buffer A and was placed at 4° C. Then, the reaction mixture was subjected to immunoprecipitation with IRS-1 antibodies. Immunocomplexes were resolved by means of SDS-PAGE and were immunoblotted with anti-PY antibodies.

### EXAMPLE 1

### Mutations in Selected Ser Residues of IRS-1 Enhances the Ability of IRS-1 to Complex with IR and to Remain Tyr Phosphorylated Following Chronic Insulin Treatment

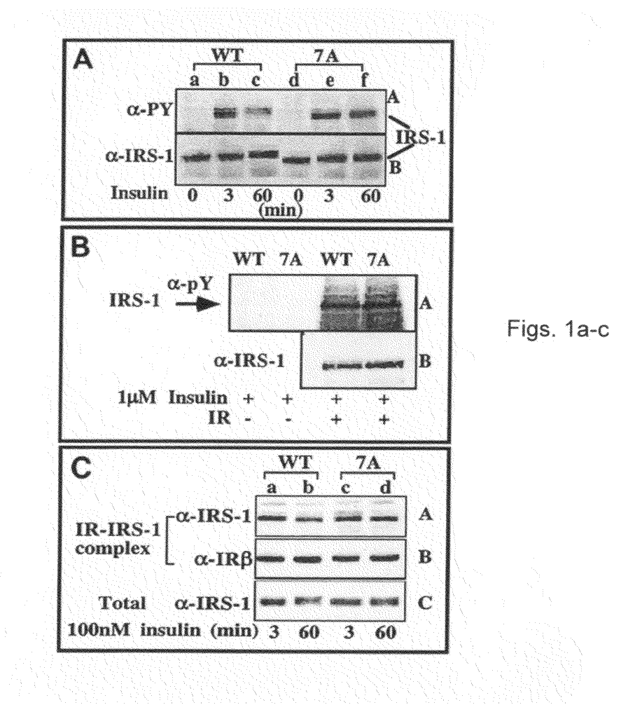

The present inventors have previously demonstrated that incubation of Fao cells with 10⁻⁷M insulin rapidly stimulates transient Tyr phosphorylation of IRS proteins, which declines following 60 min incubation with the hormone (22, 25). This decline is preceded by a decrease in the electrophoretic mobility of IRS-1 as a result of Ser/Thr phosphorylation that causes a marked (>40%) reduction in its ability to interact with the insulin receptor (22, 25). These findings, illustrated in FIG. 1a, suggest that prolonged insulin treatment, results in activation of insulin-stimulated Ser/Thr kinases that phosphorylate IRS-1, uncouple it from the receptor, and inhibit further Tyr phosphorylation of the protein. Because the PTB domain of IRS proteins is the major region that forms contacts with the juxtamembrane region of IR, phosphorylation of Ser residues located at, or in close proximity to the PTB domain might inhibit IR-IRS-1 interactions, thus negatively regulating insulin signaling.

The extended PH/PTB domain of IRS-1, termed PH/PTB-L (aa 1-430) contains 89 Ser/Thr residues, making the identification of selected phosphorylation sites within this region a serious task. To narrow down the options, seven Ser residues, located at potential phosphorylation sites for PKC, a family of Ser kinases whose conventional and atypical isoforms [e.g. PKCα and PKCζ were already implicated as potential IRS-1 kinases that negatively modulate IRS-1 function (6, 21, 22, 25)] were mutated to alanine residues (see Table 4 below). The seven mutated sites (Table 4, below) contain four resides {S265, 302, 325 and 358 (numbering based on the mouse IRS-1 sequence)} within PKB/PKC phosphorylation sites (RXRXXS); two residues (S336 and 408) within PKC phosphorylation sites (RXXS) and one site (S407) that conforms to an initiation site for phosphorylation by GSK3β (SXXS). The IRS-1 mutant was named IRS-1^{7A}.

TABLE 4*
Ser265-RPRSKSQ (SEQ ID NO: 1)
Ser302-RSRTESI (SEQ ID NO: 2)
Ser325-RVRASSD (SEQ ID NO: 3)
Ser336-MSRPASV (SEQ ID NO: 4)
Ser358-RHRGSSR (SEQ ID NO: 5)
Ser407-LFPRRSSASV (SEQ ID NO: 6)
Ser408-FPRRSSA (SEQ ID NO: 7)
*The amino acids flanking the seven Ser sites that were mutated are indicated.

In vitro Tyr phosphorylation of IRS-1^{7A} by partially-purified IR revealed that IRS-1^{7A} underwent phosphorylation to a comparable level as wild-type (WT) IRS-1 (FIG. 1a), indicating that the overall structure of IRS-1^{7A} is not altered due to the 7A mutation. When transfected into CHO-T cells, IRS-1^{7A} underwent rapid Tyr phosphorylation (P-Tyr), similar to IRS-1^{WT}, in response to acute (3 min) insulin treatment (FIG. 1a, lane b vs. e), which was consistent with the in vitro study. However, while the P-Tyr content of IRS-1^{WT} rapidly declined upon prolonged (60 min) insulin treatment, IRS-1^{7A} was significantly more resistant to Tyr dephosphorylation (FIG. 1A, lane c vs. f). Furthermore, as a consequence of its increased Ser phosphorylation, the mobility of IRS-1^{WT}, treated with insulin for 60 min, was decreased when compared with its mobility at the 3 min time point (FIG. 1A, lanes c vs. b). In contrast, IRS-1^{7A} showed only a slight decrease in its mobility at 60 min. when compared to its mobility following 3 min of insulin treatment (FIG. 1A, lane f vs. e).

To study the mechanism underlying the sustained Tyr phosphorylation of IRS-1^{7A}, in vitro binding assays of IRS-1 to IR were carried out. IRS-1^{WT} and IRS-1^{7A} showed comparable binding to IR following acute (3 min) insulin treatment (FIG. 1c, lane a vs. c). As mentioned hereinabove and consistent with previous studies (22, 25), binding of IRS-1^{WT} to IR was reduced following prolonged (60 min) insulin treatment (FIG. 1c, lane b), however such reduction was not observed in IRS-1^{7A} (FIG. 1c, lane d).

These results indicate that insulin-induced phosphorylation of serine residues mutated in IRS-1^{7A}, serves to negatively regulate IRS-1 function by impairing its ability to interact with IR. Of note, four of the serines mutated in IRS-1^{7A} conform to potential PKB (positive regulatory) sites (26), while others are potential PKC (negative regulatory) sites (22). The present results indicate that when simultaneously mutated (as in 7A), the sites associated with the negative regulation are functionally dominant over the serine sites that positively regulate IRS function.

### EXAMPLE 2

### Adenoviral Infections Enables Quantitative Expression of Myc-IRS in Fao Cells

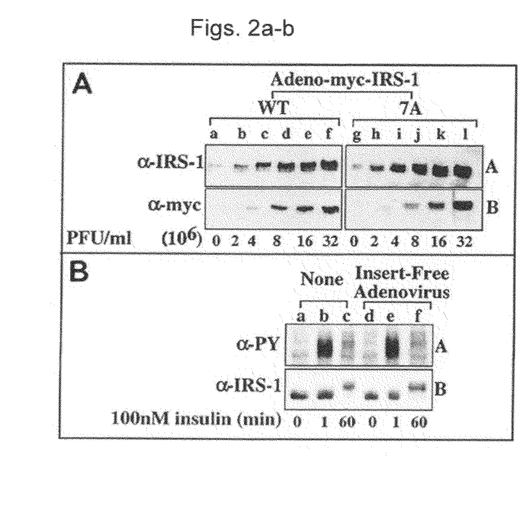

To study the effect of mutation of IRS-1 in insulin responsive cells, the cDNAs encoding Myc-tagged wild-type or mutant IRS-1^{7A} were incorporated into adenoviral constructs that allow heterologous expression in rat hepatoma Fao cells that are otherwise refractory to quantitative introduction of foreign genes by conventional transfection methods. Myc-IRS-1 (wild-type or mutant) was readily detected in Fao cells 48 h post-infection at increasing amounts that correlated with the virus titer (FIG. 2a). Of note, infection of Fao cells with control insert-free adenoviruses did not affect insulin-induced Tyr phosphorylation or the protein level of the endogenous IRS-1 (FIG. 2b), indicating that infection with adenoviruses per se does not impair insulin signaling in these cells. These results indicate this system as a good in-vitro system for studying the effect of IRS-1^{7A} on insulin signaling.

### EXAMPLE 3

### IRS-1^{7A} is Protected from the Reduction in its P-Tyr Content Following Chronic Insulin Treatment

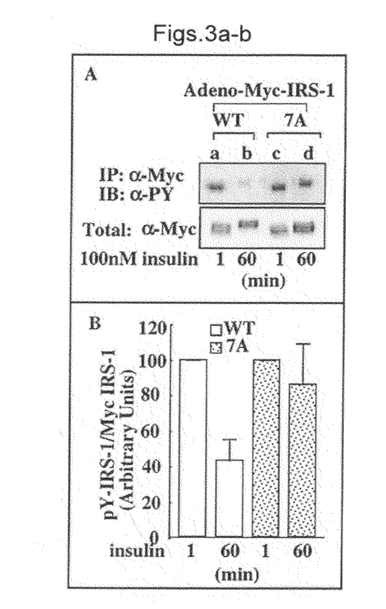

Fao cells were infected with adenoviral constructs expressing either Myc-IRS-1^{WT} or Myc-IRS-1^{7A} and their insulin-induced Tyr phosphorylation was compared. Myc-IRS-1^{7A} underwent rapid Tyr phosphorylation, similar to wild type IRS-1, in response to acute (1 min) insulin treatment (FIG. 3a. lanes a vs. c). However, while wild-type IRS-1 Tyr phosphorylation content was significantly reduced (˜55%) following prolonged (60 min) insulin treatment (FIG. 3a lanes a vs. b; FIG. 3b), IRS-1^{7A} maintained its P-Tyr content that was reduced only ˜15% following 60 min insulin treatment (FIG. 3a lanes c vs. d; FIG. 3b).

These results further indicate that mutations of potential inhibitory Ser phosphorylation sites protects IRS-1^{7A} from the action of insulin-stimulated IRS-1 kinases. This prevents the dissociation of IRS-1 from the IR (FIG. 1c) and enables it to maintain its Tyr-phosphorylated active conformation.

To determine whether IRS-1^{7A} is more resistant to proteolytic cleavage, the effects of the proteosome inhibitor MG-132 were studied. The presence of the inhibitor did not alter significantly the protein content and Tyr phosphorylation patterns of IRS-1^{7A} and IRS-1^{WT} (not shown). These findings suggest that there is no massive proteolysis of IRS-1 in the model system.

### EXAMPLE 4

### PKB and its Downstream Effectors, but not MAPK, are Activated to a Greater Extent in Fao Cells Infected with Myc-IRS-1^{7A}

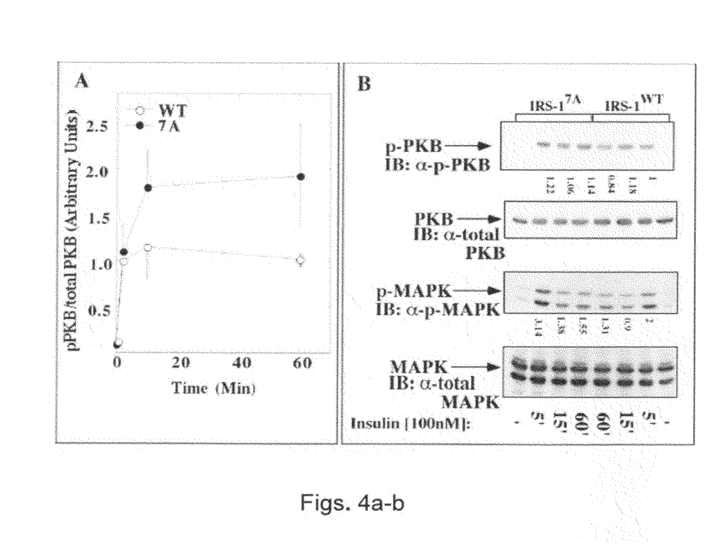

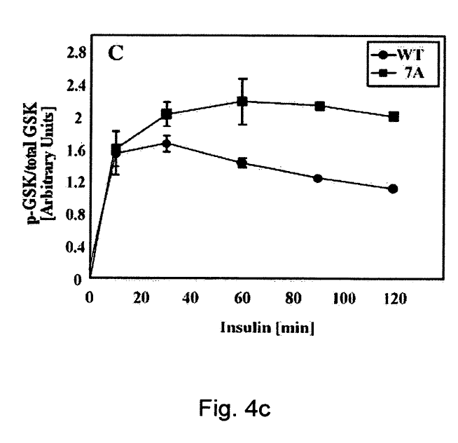

To study the effects of the 7A mutation on downstream effectors of IRS-1, PKB activity was compared in Fao cells infected either with Myc-IRS-1^{WT} or Myc-IRS-1^{7A}. PKB underwent rapid phosphorylation (on Ser408) in response to insulin, which slightly decreased following 60 min of insulin treatment in Fao cells infected with Myc-IRS-1^{WT}. As shown in FIG. 4a, this activation was potentiated 1.5-2 fold in cells infected with an equal amount of Myc-IRS-1^{7A}. In contrast, there was no difference in the extent of insulin-stimulated activation of MAPK between Fao cells infected with either IRS-1 construct (FIG. 4b).

These results suggest that the ability of IRS-1 to maintain its Tyr-phosphorylated active conformation results in more sustained activation of PKB, a downstream effector of PI3K, while MAPK activity, which is activated along the Shc/Grb2/Sos pathway is largely unaffected by the introduction of the different IRS-1 constructs. The enhanced activation of PKB was translated to a higher phosphorylation of its downstream effectors. As shown in FIG. 4c, GSK-3 underwent significantly higher (˜2-fold) phosphorylation in cells infected with Myc-IRS-1^{7A}, and similar results were observed when the phosphorylation and activation of FKHR were monitored (not shown).

### EXAMPLE 5

### Inducers of Insulin Resistance are Less Potent in Inhibiting Insulin-Stimulated Tyr Phosphorylation of IRS-1^{7A}

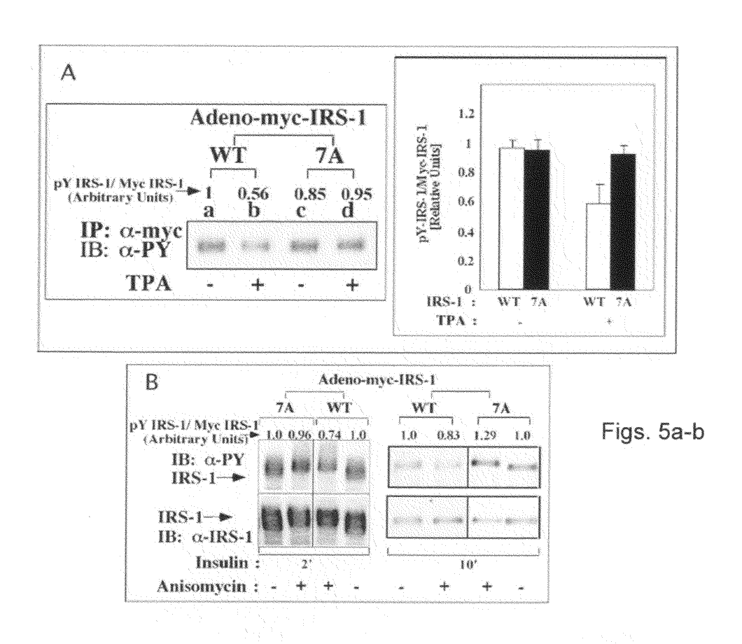

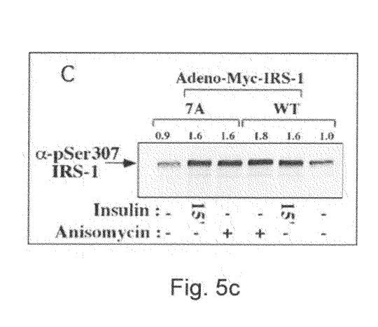

The effects of inducers of insulin resistance on Tyr phosphorylation of IRS-1^{WT} and IRS-1^{7A} were compared. Consistent with the results presented in FIGS. 3a-b, Myc-IRS-1^{7A} underwent rapid Tyr phosphorylation similar to Myc-IRS-1^{WT} in response to acute insulin treatment (FIGS. 5a-b, lane a vs. c). Pretreatment with TPA significantly inhibited (˜40%) the insulin-stimulated Tyr phosphorylation of Myc-IRS-1^{WT}, while Tyr phosphorylation of IRS-1^{7A} was affected to a much lower extent (FIG. 5a). Similar results were observed in anisomycin-treated cell (FIG. 5b). Of note, the introduction of the 7A mutation did not impair the ability of IRS-1 to undergo phosphorylation on Ser307, either as result of insulin treatment, or following treatment with inducers of insulin resistance, such as anisomycin (FIG. 5c).

IRS-1^{7A} also conferred protection against the action of free-fatty acids, the underlying cause of obesity-induced insulin resistance (32).

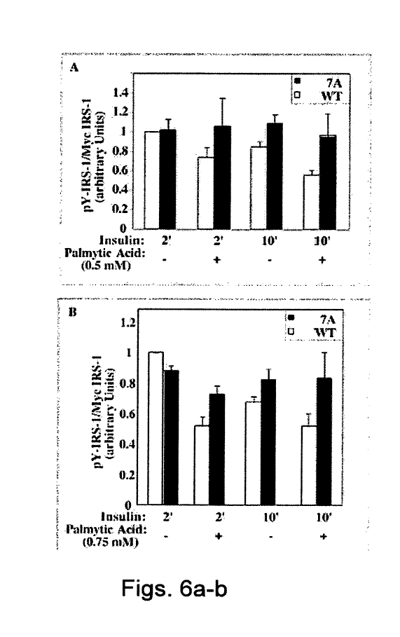

As shown in FIGS. 6a-b, pre-treatment of Fao cells with FFA (0.5-0.75 mM) reduced (˜35%) their subsequent response to insulin, as manifested by the reduction in insulin-stimulated Tyr phosphorylation of IRS-1^{WT.} However, IRS-1^{7A} was significantly more resistant to the inhibitory effects of FFA as evident by the smaller reduction in its Tyr phosphorylation state following treatment with FFA. These findings implicate Ser residues among the 7A as being targets for IRS kinases activated by inducers of insulin resistance. They further suggest that mutations of these sites might confer protection against the adverse effects of these agents.

### EXAMPLE 6

### An Extended PH/PTB Domain of IRS-1 (PH/PTB-L) Undergoes Ser Phosphorylation in vivo in Response to Chronic Insulin Treatment

To better analyze the Ser residues that are subjected to insulin-stimulated phosphorylation, a truncated form of IRS-1 (aa 1-430) encoding an extended PH/PTB domain, named PH/PTB-L, was generated and CHO-T cells that overexpress Myc-PH/PTB-L were studied.

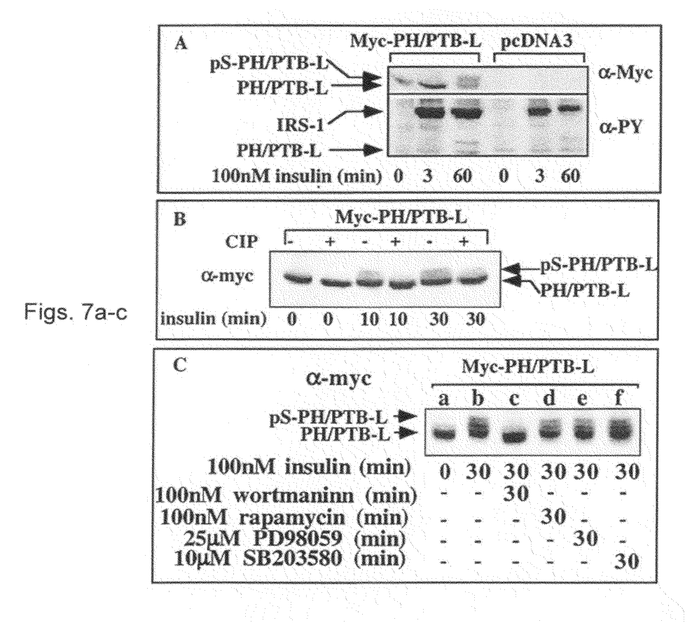

As expected, Myc-PH/PTB-L failed to undergo Tyr phosphorylation in response to insulin (FIG. 7a), because all Tyr phosphorylation sites of IRS-1 are confined to its C-terminal region which was deleted in PH/PTB-L. However, PH-PTB-L was subjected to insulin-induced Ser phosphorylation, as exemplified by its mobility shift, when CHO cells were treated with insulin for 60 min (FIG. 7a).

To ensure that the mobility shift was due to Ser phosphorylation, Myc-PH/PTB-L, isolated from insulin-treated cells, was subjected to in vitro dephosphorylation with calf intestine alkaline phosphatase (CIP). Such treatment abolished the mobility shift of PH/PTB-L, indicating that it indeed resulted from Ser phosphorylation (FIG. 7b). Pretreatment with wortmannin, a PI3K inhibitor, completely blocked the mobility shift induced by chronic insulin treatment (FIG. 7C), whereas inhibitors of mTOR (rapamycin); MEK (PD98059) and p38 MAPK (SB203580) were ineffective. These results suggest that Ser residues located within PH-PTB-L undergo phosphorylation by insulin-stimulated wortmannin-sensitive IRS kinases.

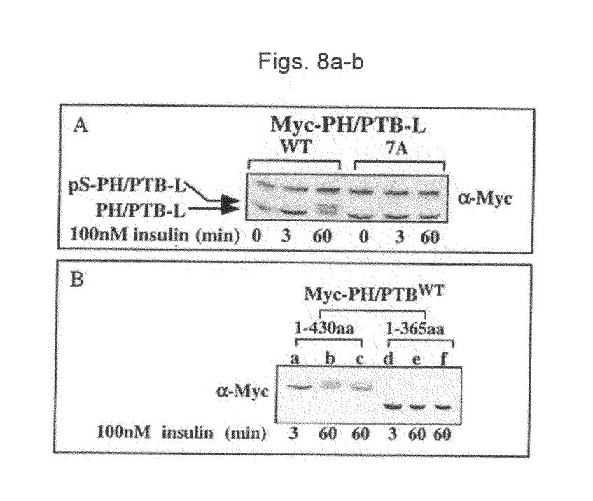

To test whether insulin-stimulated phosphorylation of PH-PTB-L takes place at Ser sites mutated in IRS-1^{7A}, a Myc-PH/PTB-L domain harboring the 7A mutation was generated. In contrast to PH/PTB-L^{WT}, PH/PTB-L^{7A} showed no mobility shift (Ser phosphorylation) following prolonged insulin treatment (FIG. 8a), indicating that some of the serines mutated in PH/PTB-L^{7A} are subjected to in vivo phosphorylation that results in decreased mobility of PH/PTB-L^{WT} in response to prolonged insulin treatment.

### EXAMPLE 7

### Truncation of PH/PTB-L, which Deletes Ser407/408, or a Selected Mutation of Ser408 Inhibits the Ability of PH/PTB-L to Undergo Ser Phosphorylation

To further analyze the Ser sites under study, a shorter truncated form of IRS-1, encoding amino acid 1-365 was generated and was named PH/PTB-S. PH/PTB-S contains five of the seven Ser residues mutated in IRS-1^{7A} but it lacks S407 and S408. When PH/PTB-S was introduced into CHO-T cells, there was no decrease in its mobility following prolonged insulin treatment (FIG. 8b), indicating that phosphorylation of Ser 407 and/or Ser408 presumably contributes to the mobility shift of PH/PTB-L, induced following prolonged insulin treatment.

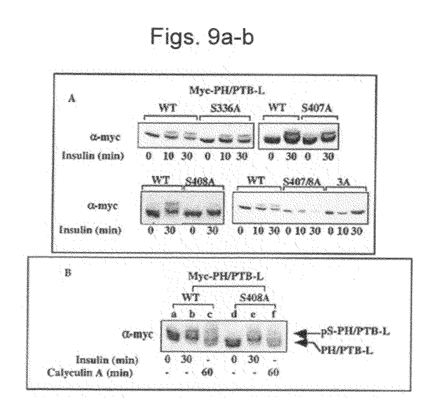

In view of these results, selected mutations of Ser407 and 408 to Ala (alone or combined) were introduced into PH/PTB-L. Additional mutation to Ala of Ser336 served as control. As shown in FIG. 9a, PH/PTB-L^{S336A} and PH/PTB-L^{S407A} manifested identical mobility-shifts as PH/PTB-L^{WT}, following 30 min insulin treatment. In contrast, mutation of Ser408, either alone (PH/PTB-L^{S408A}); together with Ser407 (PH/PTB-L^{407/S408A}), or the triple mutation of Ser336/407/408 (PH/PTB-L^{3A}) abolished the mobility shift induced upon 30 min insulin treatment. These results suggest that phosphorylation of Ser 408 significantly contributes to the decreased mobility of PH/PTB-L following prolonged insulin treatment.

Calyculin A, a Ser/Thr phosphatase inhibitor and an inducer of insulin resistance, triggers Ser phosphorylation of the full-length IRS-1 (25), as well as the phosphorylation of Myc-PH/PTB-L^{WT}, exemplified by its mobility shift (FIG. 9b). When Ser408 was mutated to Ala, the mobility shifts induced either by insulin or by calyculin A were significantly reduced (FIG. 9b, lanes b vs. e and c vs. f), suggesting that Ser408 might be a target for Ser kinases activated either by insulin, or by selected inducers of insulin resistance.

### EXAMPLE 8

### Ser408 is a Target for in vivo Phosphorylation Promoted Either by Insulin or by Inducers of Insulin Resistance

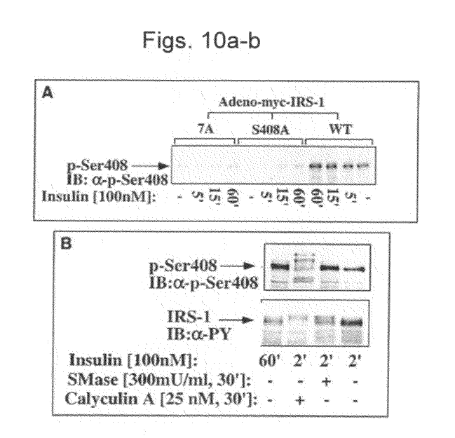

To ensure that Ser408 is indeed an in vivo phosphorylation site of IRS-1, antibodies directed against the phosphorylated form of Ser408 (anti-P-S408) were generated. As shown in FIG. 10a, these antibodies readily reacted with IRS-1^{WT}, but they failed to interact either with IRS-1^{7A} or with IRS-1^{S408A}, expressed in Fao cells as adenoviral construct. To determine whether S408 is subjected to in vivo phosphorylation triggered by inducers of insulin resistance, Fao cells were infected with IRS-1^{WT} and were then incubated with different inducers of insulin resistance. As shown in FIG. 10b, SMase readily induced in vivo phosphorylation of S408, comparable to that induced by 60 min insulin treatment. Similarly, Calyculin A induced phosphorylation of S408 that was accompanied by a significant mobility shift of the protein. These findings clearly indicate that S408 is an in vivo target for phosphorylation induced either by prolonged insulin treatment or by inducers of insulin resistance.

Of note, insulin-induced phosphorylation of Ser408 occurred at a slower rate than the Tyr phosphorylation of IRS-1. While maximal Tyr phosphorylation of IRS-1 was already detected following 2 min insulin treatment (FIG. 1a), the maximal phosphorylation of Ser408 required 30-60 min incubation with the hormone (FIG. 10a). These findings are consistent with the hypothesis that negative-feedback control mechanisms that involve Ser408 phosphorylation are triggered by insulin only subsequent to the induction of its signaling cascades which involves Tyr phosphorylation of IRS proteins.

### EXAMPLE 9

### Mutation of Ser408 Confers IRS-1 Partial Protection from the Inhibitory Action of Inducers of Insulin Resistance

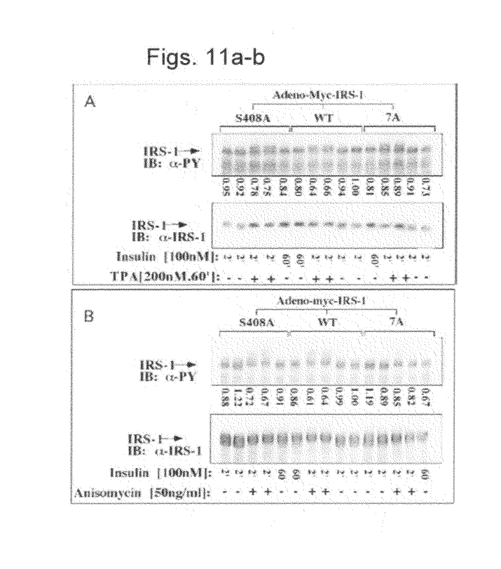

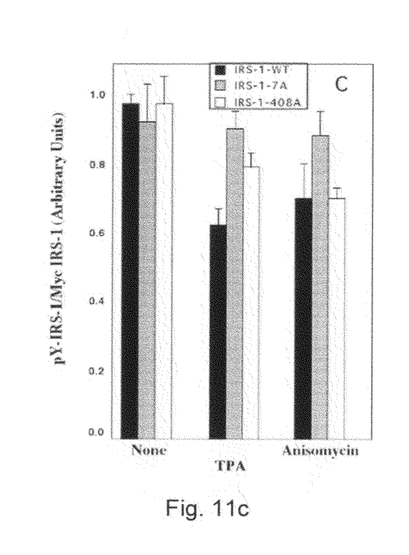

To study the effects of the S408A mutation on IRS-1 function, Fao cells were infected with adenoviral constructs expressing either Myc-IRS-1^{WT}, Myc-IRS-1^{7A} or Myc-IRS-1^{408A} and their insulin-induced Tyr phosphorylation was compared. Myc-IRS-1^{408A} underwent rapid Tyr phosphorylation similar to IRS-1^{WT} or IRS-1^{7A} in response to acute insulin treatment (FIG. 11). However, prolonged (60 min) insulin treatment reduced to a similar extent the Tyr phosphorylation level of either wild-type IRS-1 or its 408A mutant, while the Tyr phosphorylation state of the 7A mutant remained elevated. Yet, the inhibitory effects TPA on insulin-stimulated Tyr phosphorylation of IRS-1 could be partially prevented in the IRS-1^{408A} mutant (FIG. 11A), although IRS-1^{408A} could not protect against the inhibitory effects of anisomycin (FIGS. 11b and c). These results suggest that mutation of Ser408 might confer protection against selected inducers of insulin resistance, while mutation of additional sites among the 7S is required to confer protection from the inhibitory action of prolonged insulin treatment and a wider spectrum of inducers of insulin resistance.

### Discussion

IRS proteins are key players in propagating insulin signaling and are therefore subjected to feedback regulatory systems that inhibit their action. Feed-back regulation involves phosphatase-mediated dephosphorylation (8) or Ser/Thr phosphorylation of functionally active Tyr-phosphorylated IRS proteins (40). Ser/Thr phosphorylation can induce, for example, the dissociation of the IRS proteins from the insulin receptor or from downstream effectors, or can lead to their degradation (40). The above results substantiate that Ser phosphorylation might interfere with the association of IRS-1 with IR and negatively regulate IRS-1 function. Since the PTB domain of IRS-1 mediates its interactions with the juxtamembrane region of IR, the present inventors hypothesized that phosphorylation of a distinct set of Ser residues located within or proximal to the PTB domain may interrupt with IR-IRS-1 complex formation. The present inventors have previously shown that phosphorylation of IRS-1, induced by PKCζ impairs its ability to interact with IR and undergo Tyr phosphorylation (22), therefore, seven Ser residues at or in close proximity to the PTB domain, six being at potential PKC phosphorylation sites, were mutated to Ala. Such an IRS-1 mutant, named IRS-1^{7A}, failed to undergo Ser phosphorylation following prolonged insulin treatment. The resistance to Ser phosphorylation enabled the mutant IRS-1 to remain tightly complexed with the IR, and to maintain its active Tyr phosphorylated state that is otherwise impaired in wild-type IRS-1 as a result of Ser phosphorylation following prolonged insulin treatment. As a consequence of its sustained Tyr phosphorylation, IRS-1^{7A} can better propagate insulin signaling manifested by its ability to maintain sustained activation of PKB and its downstream effectors, GSK-β and FKHR (3).

These results therefore suggest that at least some of the mutated Ser residues are subjected to phosphorylation by insulin-stimulated IRS kinases that negatively regulate IRS-1 function. The IRS-1^{7A} mutant was also protected from inducers of insulin resistance, such as TPA, anisomycin and free-fatty acids that utilize this physiological feedback control mechanism to promote phosphorylation of IRS-1 (wild-type) at the same Ser sites, and in such a way inhibit its action. Hence, the seven Ser residues subjected to mutation, denoted 7S, are key sites for the regulation of IRS-1 function because they serve as a point of convergence, where physiological feed back control mechanisms, triggered by insulin-stimulated IRS kinases, overlap with IRS kinases triggered by inducers of insulin resistance that phosphorylate the very same sites, and in such a way terminate insulin signaling.

Several lines of evidence support this conclusion. First, it was demonstrated that mutation to Ala of the seven Ser sites (S265, 302, 325, 336, 358 407 and 408) of IRS-1 obliterates its mobility shift, a characteristic feature of Ser-phosphorylated IRS-1. Failure of IRS-1^{7A} to undergo insulin-stimulated Ser phosphorylation cannot be attributed to a major conformational change introduced by the mutations per se because IRS-1^{7A} undergoes in vitro Tyr phosphorylation by IR to a similar extent as the wild-type protein. Furthermore, both wild-type IRS-1 and its 7A mutant are subjected to comparable levels of in vivo Tyr phosphorylation following acute insulin treatment, again indicating that the 7A mutation does not impair the ability of IRS-1^{7A} to localize in close proximity to the IR. In an attempt to identify the Ser residues subjected to phosphorylation, truncated forms of IRS-1 that either contains all (PH/PTB-L, aa 1-430) or only five (PH/PTB-S, aa 1-365) of the seven serines under study were generated. Like the full-length IRS-1, PH/PTB-L was subjected to insulin-stimulated Ser/Thr phosphorylation that was evident by its reduced mobility following chronic insulin treatment. The contribution of the 7S sites to the mobility shift became evident when it was shown that PH/PTB-L^{7A} fails to undergo a mobility shift following insulin stimulation. Furthermore, only PH/PTB-L but not the PH/PTB-S, demonstrated a mobility shift, suggesting that the Ser residues involved are 5407 or/and S408. Indeed, introduction of single mutations into PH/PTB-L indicated that Ser 408 is the prime site within the PH/PTB-L^{WT} that contributes to the mobility shift in response to prolonged insulin treatment.

Ser408 is a genuine in vivo phosphorylation site. This conclusion is based upon the fact that its phosphorylation following insulin treatment can be detected using peptide-specific antibodies that selectively recognize the phosphorylated form of S408. Of note, phosphorylation of S408 occurs at a slower rate than the Tyr phosphorylation rate of IRS-1. This finding is in accordance with the concept that Ser phosphorylation of IRS proteins, being a mean to terminate insulin action, should commence subsequent to Tyr phosphorylation of the IRS proteins, which triggers insulin signalling.

Insulin-stimulated phosphorylation of S408 is wortmannin-sensitive, suggesting that the S408 kinase is a downstream effector of PI3K. This kinase presumably differs from mTOR because rapamycin fails to inhibit phosphorylation of S408. Because S408 is located at a PKC phosphorylation site (RXXS), a potential S408 kinases could be PKCζ. Indeed, previous studies by us (22) and by others (28) have indicated that IRS-1 is subjected to Ser phosphorylation by PKCζ, which is a downstream effector of insulin along the PI3K pathway (22). Several inducers of insulin resistance such as TNFα (30) and fatty acids (37) can also promote the activation of PKCζ, turning it into a common kinase being activated by physiological and pathological signals to promote Ser phosphorylation of IRS-1.

Still, phosphorylation of S408 might be necessary but it is certainly insufficient to inhibit Tyr phosphorylation of IRS-1 and uncouple IR-IRS-1 complexes. This conclusion is based upon the fact that introduction into Fao cells of adenoviral construct that harbors the S408A mutation, fails to protect IRS-1^{S408A} from undergoing Tyr dephosphorylation following chronic insulin treatment. This contrasts with the full protection against Tyr dephosphorylation provided by the IRS-1^{7A} mutant. Similarly, while IRS-1^{S408A} is protected from the inhibitory effects exerted by TPA, it fails to resist other inhibitors of insulin-induced Tyr phosphorylation. This differs from the almost complete protection against several inducers of insulin resistance (e.g. TPA, FFA, anisomycin) provided by the 7A mutant. These findings suggest that S408 might be target for phosphorylation by IRS kinases activated only by selected inducers of insulin resistance. Hence, additional sites among the 7S, acting in concert with S408, may function as ‘inhibitory’ Ser phosphorylation sites for IRS function. Their phosphorylation could be catalyzed by PKCζ or by other IRS kinases, which are downstream effectors of PI3K. A potential candidate could be IKK\tilde{β} IKKβ is a Ser/Thr kinase that is part of the IKK complex that phosphorylates the inhibitor of NF-κB, IκB. IKKβ can bind PKCζ both in vitro and in vivo, it serves as an in vitro substrate for PKCζ and it is activated by a functional PKCζ (18). IKKβ is activated by insulin in Fao rat hepatoma cells², furthermore, insulin-stimulated Ser phosphorylation of IRS-1 is inhibited by salicylates, implicating IKKβ as an insulin-stimulated IRS kinase². Indeed, IRS-1 is a direct substrate of stress-activated IKKβ that phosphorylate IRS-1 on Ser 312 (the human homologue of mouse Ser 307) (9) and could phosphorylate additional sites as well. It therefore appears that PI3K controls a couple of Ser/Thr kinases to negatively regulate IRS-1 function; mTOR acting at the C-terminal tail and PKCζ/IKKβ acting at the N-terminal region.

Paz and co-workers have previously shown that mutations of four Ser residues (4S) (S265, 302, 325 and 358), among the 7S, which are located at PKB/PKC phosphorylation sites (RXRXXS), renders IRS-1 prone to the action of PTPs, thus implicating some of these 4S residues as positive regulators of IRS-1 function (26). This conclusion is supported by the fact that overexpression of PKB significantly attenuates the rate of Tyr dephosphorylation of IRS-1 following 60 min. of treatment with insulin (26). Here it is shown that mutation of seven Ser sites which include those 4S ‘positive sites’ exert an overall protective effect on IRS-1 functions, suggesting that mutation of the ‘negative sites’ among the 7S, as in IRS-1^{7A}, functionally dominates the mutation of the ‘positive sites’ as in IRS-1^{4A}. Hence, distinct arrays of Ser residues might either negatively or positively regulate IRS protein function. Under physiological condition, upon insulin stimulation, the ‘positive sites’ must be phosphorylated prior to the ‘negative sites’, to enable the insulin signal to be first potentiated before it is being attenuated as part of a negative feedback loop. Under pathological condition, inducers of insulin resistance presumably trigger the phosphorylation of only the ‘negative’ sites, with no effects on the ‘positive’ sites, thus preventing the propagation of insulin signals mediated by IRS proteins and thus causing insulin resistance.

Several other Ser sites, different altogether from the 7S, were already implicated in the negative regulation of IRS-1 function. ‘Conventional’ members of the PKC family which are activated by phorbol esters or endothelin-1, activate members of the MAPK pathway to phosphorylate IRS-1 at Ser612 and at additional sites in its COOH tail (5). Such phosphorylation inhibits the interactions of IRS-1 both with IR and with downstream effectors of IRS-1, such as PI3K. Hence, phosphorylation of Ser residues at the COOH tail of IRS-1, could synergize with phosphorylation at its N-terminal regions. This is in accordance with the idea that increased levels of regulatory inputs can provide a more subtle and powerful regulation. Still, most relevant to this study is Ser307 within the PTB domain, the phosphorylation of which negatively regulates IRS functions (1, 10, 39). Phosphorylation of S307 is catalyzed by a number of kinases, some, like JNK, are activated by insulin (1, 2, 19). IRS-1^{7A} contains an intact Ser307, yet it is resistant to the inhibitory effects of chronic insulin treatment or to the action of inducers of insulin resistance. The present findings rule out the possibility that introduction of the 7A mutation impairs the phosphorylation of Ser307, therefore it can suggested that phosphorylation of Ser307 is not sufficient to impair IRS-1 function, and phosphorylation of additional Ser sites, Ser408 and others among the 7S, is required to uncouple IR-IRS-1 complexes and inhibit IRS-1 functions.

Ser/Thr phosphorylation can inhibit IRS-1 function in a number of ways (4, 12, 15, 22, 23, 25, 27, 34). But, regardless of mechanism, increased Ser phosphorylation of IRS-1 at ‘inhibitory’ sites underlies a key mode to inhibit IRS proteins function, utilized either by insulin itself, as a physiological negative feed-back control mechanism, or by different agents that induce insulin resistance and type 2 Diabetes. Given the large number of stimuli, pathways, kinases, and potential sites involved, it appears that Ser/Thr phosphorylation of IRS proteins represents a combinatorial consequence of several kinases activated by different pathways, acting in concert to phosphorylate multiple sites. Devising effective means to prevent the phosphorylation of the ‘inhibitory’ sites could turn beneficial in attempts to promote insulin action and protect against the adverse effects of inducers of insulin resistance.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

### REFERENCES

### Other References are Cited in the Text

1. Aguirre, V., T. Uchida, L. Yenush, R. Davis, and M. F. White. 2000. The c-Jun NH(2)-terminal kinase promotes insulin resistance during association with insulin receptor substrate-1 and phosphorylation of Ser(307). J Biol Chem 275:9047-9054.
2. Aguirre, V., E. D. Werner, J. Giraud, Y. H. Lee, S. E. Shoelson, and M. F. White. 2002. Phosphorylation of Ser307 in insulin receptor substrate-1 blocks interactions with the insulin receptor and inhibits insulin action. J Biol Chem 277:1531-7.
3. Brunet, A., A. Bonni, M. J. Zigmond, M. Z. Lin, P. Juo, L. S. Hu, M. J. Anderson, K. C. Arden, J. Blenis, and M. E. Greenberg. 1999. Akt promotes cell survival by phosphorylating and inhibiting a Forkhead transcription factor. Cell 96:857-868.
4. Clark, S. F., J. C. Molero, and D. E. James. 2000. Release of insulin receptor substrate proteins from an intracellular complex coincides with the development of insulin resistance. J Biol Chem 275:3819-3826.
5. De Fea, K., and R. A. Roth. 1997. Modulation of insulin receptor substrate-1 tyrosine phosphorylation and function by mitogen-activated protein kinase. J Biol Chem 272:31400-31406.
6. De Fea, K., and R. A. Roth. 1997. Protein kinase C modulation of insulin receptor substrate-1 tyrosine phosphorylation requires serine 612. Biochemistry 36:12939-12947.
7. Eck, M. J., P. S. Dhe, T. Trub, R. T. Nolte, and S. E. Shoelson. 1996. Structure of the IRS-1 PTB domain bound to the juxtamembrane region of the insulin receptor. Cell 85:695-705.
8. Elchebly, M., P. Payette, E. Michaliszyn, W. Cromlish, S. Collins, A. L. Loy, D. Normandin, A. Cheng, H. J. Himms, C. C. Chan, C. Ramachandran, M. J. Gresser, M. L. Tremblay, and B. P. Kennedy. 1999. Increased insulin sensitivity and obesity resistance in mice lacking the protein tyrosine phosphatase-1B gene. Science 283:1544-1548.
9. Gao, Z., D. Hwang, F. Bataille, M. Lefevre, D. York, M. Quon, and J. Ye. 2002. Serine phosphorylation of insulin receptor substrate 1 (IRS-1) by inhibitor KappaB kinase (IKK) complex. J Biol Chem 277:48115-48121.
10. Gao, Z., A. Zuberi, M. J. Quon, Z. Dong, and J. Ye. 2003. Aspirin inhibits serine phosphorylation of IRS-1 in TNF-treated cells through targeting multiple serine kinases. J Biol Chem In Press.
11. Hadari, Y. R., K. Paz, R. Dekel, T. Mestrovic, D. Accili, and Y. Zick. 1995. Galectin-8. A new rat lectin, related to galectin-4. J Biol Chem 270:3447-53.
12. Haruta, T., T. Uno, J. Kawahara, A. Takano, K. Egawa, P. M. Sharma, J. M. Olefsky, and M. Kobayashi. 2000. A rapamycin-sensitive pathway down-regulates insulin signaling via phosphorylation and proteasomal degradation of insulin receptor substrate-1. Mol Endocrinol 14:783-794.
13. He, T. C., S. Zhou, L. T. da Costa, J. Yu, K. W. Kinzler, and B. Vogelstein. 1998. A simplified system for generating recombinant adenoviruses. Proc. Natl. Acad. Sci. USA 95:2509-2514.
14. Hemi, R., K. Paz, N. Wertheim, A. Karasik, Y. Zick, and H. Kanety. 2002. Transactivation of ErbB2 and ErbB3 by tumor necrosis factor-alpha and anisomycin leads to impaired insulin signaling through serine/threonine phosphorylation of IRS proteins. J Biol Chem 277:8961-8969.
15. Hotamisligil, G. S., P. Peraldi, A. Budavari, R. Ellis, M. F. White, and B. M. Spiegelman. 1996. IRS-1-mediated inhibition of insulin receptor tyrosine kinase activity in TNF-alpha- and obesity-induced insulin resistance. Science 271:665-668.
16. Khan, A. H., and J. E. Pessin. 2002. Insulin regulation of glucose uptake: a complex interplay of intracellular signalling pathways. Diabetologia 45:1475-1483.
17. Laemmli, U. K. 1970. Cleavage of structural proteins during the assembly of the head of bacteriophage T4. Nature 227:680-685.
18. Lallena, M.-J., M. T. Diaz-Meco, G. Bren, C. V. Paya, and J. Moscat. 1999. Activation of IkappaB kinase beta by protein kinase C isoforms. Mol Cell Biol 19:2180-2188.
19. Lee, Y.-H., J. Giraud, R. J. Davis, and M. F. White. 2003. cJUN N-terminal Kinase (JNK) mediates feedback inhibition of the insulin signaling cascade. J. Biol. Chem. 278:2896-2002.
20. LeRoith, D., and Y. Zick. 2001. Recent Advances in our understanding of insulin action and insulin resistance. Diabetes Care 24:588-597.
21. Li, J., K. DeFea, and R. A. Roth. 1999. Modulation of insulin receptor substrate-1 tyrosine phosphorylation by an Akt/phosphatidylinositol 3-kinase pathway. J Biol Chem 274:9351-9356.
22. Liu, Y. F., K. Paz, A. Herschkovitz, A. Alt, T. Tennenbaum, S. R. Sampson, M. Ohba, T. Kuroki, D. LeRoith, and Y. Zick. 2001. Insulin stimulates PKCzeta-mediated phosphorylation of insulin receptor substrate-1 (IRS-1). A self-attenuated mechanism to negatively regulate the function of IRS proteins. J Biol Chem 276:14459-14465.
23. Mothe, I., and E. Van Obberghen. 1996. Phosphorylation of insulin receptor substrate-1 on multiple serine residues, 612, 632, 662, and 731, modulates insulin action. J Biol Chem 271:11222-11227.
24. Pawson, T. 1995. Protein modules and signalling networks. Nature 373:573-580.
25. Paz, K., R. Hemi, R. LeRoith, A. Karasik, E. Elhanany, H. Kanety, and Y. Zick. 1997. A Molecular Basis for Insulin Resistance: Elevated Serine/Threonine Phosphorylation of IRS-1 and IRS-2 Inhibits their Binding to the Juxtamembrane Region of the Insulin Receptor and Impairs Their Ability to Undergo Insulin-Induced Tyrosine Phosphorylation. J. Biol. Chem. 272:29911-29918.
26. Paz, K., Yan-Fang, L., Shorer, H., Hemi R., LeRoith, D., Quon, M., Kanety, H., Seger, R., and Zick, Y. 1999. Phosphorylation of Insulin Receptor Substrate-1 (IRS-1) by PKB Positively Regulates IRS-1 Function. J. Biol. Chem 274:28816-28822.
27. Pederson, T. M., D. L. Kramer, and C. M. Rondinone. 2001. Serine/threonine phosphorylation of IRS-1 triggers its degradation: possible regulation by tyrosine phosphorylation. Diabetes 50:24-31.
28. Ravichandran, L. V., D. L. Esposito, J. Chen, and M. J. Quon. 2001. PKC-{zeta} phosphorylates IRS-1 and impairs its ability to activate PI 3-kinase in response to insulin. J Biol Chem 276:3543-3549.
29. Saltiel, A. R., and J. E. Pessin. 2002. Insulin signaling pathways in time and space. Trends Cell Biol 12:65-71.
30. Sanz, L., P. Sanchez, M. J. Lallena, M. M. Diaz, and J. Moscat. 1999. The interaction of p62 with RIP links the atypical PKCs to NF-kappaB activation. EMBO J 18:3044-3053.
31. Sawka, V. D., D. S. Tartare, M. F. White, and 0. E. Van. 1996. Insulin receptor substrate-2 binds to the insulin receptor through its phosphotyrosine-binding domain and through a newly identified domain comprising amino acids 591-786. J Biol Chem 271:5980-5983.
32. Shulman, G. I. 1999. Cellular mechanisms of insulin resistance in humans. Am J Cardiol 84:3J-10J.
33. Sun, X. J., P. Rothenberg, C. R. Kahn, J. M. Backer, E. Araki, P. A. Wilden, D. A. Cahill, B. J. Goldstein, and M. F. White. 1991. Structure of the insulin receptor substrate IRS-1 defines a unique signal transduction protein. Nature 352:73-77.
34. Tirosh, A., R. Potashnik, N. Bashan, and A. Rudich. 1999. Oxidative stress disrupts insulin-induced cellular redistribution of insulin receptor substrate-1 and phosphatidylinositol 3-kinase in 3T3-L1 adipocytes. A putative cellular mechanism for impaired protein kinase B activation and GLUT4 translocation. J Biol Chem 274:10595-10602.
35. Voliovitch, H., Schindler, D., Hadari, Y. R., Taylor, S. I., Accili, D., and Zick, Y. 1995. The pleckstrin-homology (PH) domain of insulin receptor substrate-1 (IRS-1) is required for proper interaction of IRS-1 with the insulin receptor. J. Biol. Chem. 270:18083-18087.
36. Wolf, G., Trüb, T., Ottinger, E., Groninga, L., Lynch, A., White, M. F., Miyazaki, M., Lee, J., and Shoelson, S. E. 1995. PTB Domains of IRS-1 and Shc Have Distinct but Overlapping Binding Specificities. J. Biol. Chem. 270:27407-27410.
37. Wrede, C. E., L. M. Dickson, M. K. Lingohr, I. Briaud, and C. J. Rhodes. 2003. Fatty acid and phorbol ester-mediated interference of mitogenic signaling via novel protein kinase C isoforms in pancreatic beta-cells (INS-1). J Mol Endocrinol 30:271-86.
38. Wrede, C. E., L. M. Dickson, M. K. Lingohr, I. Briaud, and C. J. Rhodes. 2002. Protein kinase B/Akt prevents fatty acid-induced apoptosis in pancreatic beta-cells (INS-1). J Biol Chem 277:49676-84.
39. Yu, C., Y. Chen, H. Zong, Y. Wang, R. Bergeron, J. K. Kim, G. W. Cline, S. W. Cushman, G. J. Cooney, B. Atcheson, M. F. White, E. W. Kraegen, and G. I. Shulman. 2002. Mechanism by which fatty acids inhibit insulin activation of IRS-1 associated phosphatidylinositol 3-kinase activity in muscle. J Biol Chem 277:50230-50236.
40. Zick, Y. 2001. Insulin Resistance: a Phosphorylation-Based Uncoupling of Insulin Signaling. Trends Cell Biol. 11:437-441.

What is claimed is:

1. A method of treating insulin resistance in a subject, the method comprising administering to the subject a therapeutically effective amount of an agent capable of down-regulating phosphorylation of an IRS protein at at least one serine residue corresponding to amino acid 341, 412 and/or 413 of human IRS-1, thereby treating the insulin resistance in the subject.

2. The method of claim 1, wherein the insulin resistance is associated with a disease or condition selected from the group consisting of Type II diabetes, obesity, hyperglycemia and hyperlipidemia.

3. The method of claim 1, wherein said agent is selected from the group consisting of:
(i) a peptide comprising an IRS amino acid sequence not exceeding 50 amino acids in length which comprises said at least one serine residue;
(ii) an isolated polynucleotide which comprises a nucleic acid sequence encoding an IRS protein which comprises a mutation in said at least one serine residue; and
(iii) cells expressing said isolated polynucleotide of (ii).

4. The method of claim 1, wherein said IRS protein is selected from the group consisting of IRS-1, IRS-2 and IRS-4.

5. A peptide comprising an IRS amino acid sequence not exceeding 50 amino acids in length which comprises at least one serine residue corresponding to amino acid 341, 412 and/or 413 of human IRS-1.

6. An antibody comprising an antigen recognition domain capable of specifically binding an IRS protein phosphorylated on at least one serine residue corresponding to amino acid 341, 412 and/or 413 of human IRS-1 but does not bind said IRS protein when not phosphorylated on this respective position.

7. An isolated polynucleotide comprising a nucleic acid sequence encoding an IRS protein which comprises a mutation in at least one serine residue corresponding to amino acid 341, 412 and/or 413 of human IRS-1.

8. A nucleic acid construct comprising the isolated polynucleotide of claim 7.

9. A cell comprising the isolated polynucleotide of claim 7.

10. A method of diagnosing insulin resistance in a subject, the method comprising detecting in a biological sample of the subject, presence, absence or level of an IRS protein phosphorylated on at least one serine residue corresponding to amino acid 341, 412 and/or 413 of human IRS-1, wherein said presence or level of said phosphorylated IRS protein is indicative of said insulin resistance in the subject.

11. The method of claim 10, wherein said detecting said presence, absence or level of phosphorylated IRS protein is effected via the antibody of claim 6.

12. The method of claim 10, wherein said biological sample is a blood sample, a liver sample and/or an adipose tissue derived sample.

13. A method of identifying agents suitable for treating insulin resistance, the method comprising:
(a) contacting a biological sample comprising an IRS phosphorylated on at least one serine residue corresponding to amino acid 341, 412 and/or 413 of human IRS-1 with a plurality of agents; and
(b) identifying at least one agent of said plurality of agents being capable of dephosphorylating said at least one serine residue, thereby identifying agents suitable for treating insulin resistance.

14. A kit for diagnosing insulin resistance in a subject, the kit comprising a packaging material packaging the antibody of claim 6.

15. A pharmaceutical composition comprising as an active ingredient the isolated polynucleotide of claim 7 and a pharmaceutically acceptable carrier or diluent.

16. A pharmaceutical composition comprising as an active ingredient the isolated polynucleotide of claim 8 and a pharmaceutically acceptable carrier or diluent.

17. A pharmaceutical composition comprising as an active ingredient the cell of claim 9 and a pharmaceutically acceptable carrier or diluent.

18. Use of an agent capable of down-regulating phosphorylation of an IRS protein at at least one serine residue corresponding to amino acid 341, 412 and/or 413 of human IRS-1 as a pharmaceutical.

19. The use of claim 18, wherein said agent is selected from the group consisting of:
(i) a peptide comprising an IRS amino acid sequence not exceeding 50 amino acids in length which comprises said at least one serine residue;
(ii) an isolated polynucleotide which comprises a nucleic acid sequence encoding an IRS protein which comprises a mutation in said at least one serine residue; and
(iii) cells expressing said isolated polynucleotide of (ii).

20. Use of an agent capable of down-regulating phosphorylation of an IRS protein at at least one serine residue corresponding to amino acid 341, 412 and/or 413 of human IRS-1 for the manufacture of a medicament identified for treating insulin resistance.

21. The use of claim 19, wherein said agent is selected from the group consisting of:
(i) a peptide comprising an IRS amino acid sequence not exceeding 50 amino acids in length which comprises said at least one serine residue;
(ii) an isolated polynucleotide which comprises a nucleic acid sequence encoding an IRS protein which comprises a mutation in said at least one serine residue; and
(iii) cells expressing said isolated polynucleotide of (ii).


SEQUENCE LISTING (21 sequences)

SEQ ID NO: 1 - protein, 7 aa, Artificial sequence
  A peptide containing IRS Ser phosphorylation site and flanking amino acid residues
RPRSKSQ

SEQ ID NO: 2 - protein, 7 aa, Artificial sequence
  A peptide containing IRS Ser phosphorylation site and flanking amino acid residues
RSRTESI

SEQ ID NO: 3 - protein, 7 aa, Artificial sequence
  A peptide containing IRS Ser phosphorylation site and flanking amino acid residues
RVRASSD

SEQ ID NO: 4 - protein, 7 aa, Artificial sequence
  A peptide containing IRS Ser phosphorylation site and flanking amino acid residues
MSRPASV

SEQ ID NO: 5 - protein, 7 aa, Artificial sequence
  A peptide containing IRS Ser phosphorylation site and flanking amino acid residues
RHRGSSR

SEQ ID NO: 6 - protein, 10 aa, Artificial sequence
  A peptide containing IRS Ser phosphorylation site and flanking amino acid residues
LFPRRSSASV

SEQ ID NO: 7 - protein, 7 aa, Artificial sequence
  A peptide containing IRS Ser phosphorylation site and flanking amino acid residues
FPRRSSA

SEQ ID NO: 8 - protein, 13 aa, Artificial sequence
  A peptide containing IRS Ser phosphorylation site flanking amino acid residues and an additional Cys residue at the N′
CLFPRRSSASVSG

SEQ ID NO: 9 - protein, 16 aa, Artificial sequence
  Myc tag and the missing amino-acids of IRS-1 added to the IRS-1 construct
MEQKLISEEDLNMASP

SEQ ID NO: 10 - DNA, 27 bp, Artificial sequence
  Single strand DNA oligonucleotide
caggatccgcatatggaacaaaagctc

SEQ ID NO: 11 - DNA, 27 bp, Artificial sequence
  Single strand DNA oligonucleotide
gagaattcatcagggactagaaccata

SEQ ID NO: 12 - DNA, 23 bp, Artificial sequence
  Single strand DNA oligonucleotide
aagcttaagatatcgatcatatg

SEQ ID NO: 13 - DNA, 24 bp, Artificial sequence
  Single strand DNA oligonucleotide
ttagttgagtggggggtgcagcct

SEQ ID NO: 14 - DNA, 30 bp, Artificial sequence
  Single strand DNA oligonucleotide
catgtcccgtccagctgcagtggatggcag

SEQ ID NO: 15 - DNA, 30 bp, Artificial sequence
  Single strand DNA oligonucleotide
ctgccatccactgcagctggacgggacatg

SEQ ID NO: 16 - DNA, 33 bp, Artificial sequence
  Single strand DNA oligonucleotide
cttcccgaggcgcgctagcgcttccgtgtccgg

SEQ ID NO: 17 - DNA, 33 bp, Artificial sequence
  Single strand DNA oligonucleotide
ccggacacggaagcgctagcgcgcctcgggaag

SEQ ID NO: 18 - DNA, 33 bp, Artificial sequence
  Single strand DNA oligonucleotide
cttcccgaggcgctcagctgcttccgtgtccgg

SEQ ID NO: 19 - DNA, 33 bp, Artificial sequence
  Single strand DNA oligonucleotide
ccggacacggaagcagctgagcgcctcgggaag

SEQ ID NO: 20 - DNA, 33 bp, Artificial sequence
  Single strand DNA oligonucleotide
cttcccgaggcgcgctgcagcttccgtgtccgg

SEQ ID NO: 21 - DNA, 33 bp, Artificial sequence
  Single strand DNA oligonucleotide
ccggacacggaagctgcagcgcgcctcgggaag